(19) United States
(12) Patent Application Publication
(10) Pub. No.: US 2024/0304331 A1
(43) Pub. Date: Sep. 12, 2024

(54) METHOD FOR PROVIDING INFORMATION RELATED TO TYPE OF CANCER, SYSTEM FOR PROVIDING INFORMATION RELATED TO TYPE OF CANCER

(71) Applicants: Kagami Inc., Osaka (JP); National Institutes of Biomedical Innovation, Health and Nutrition, Osaka (JP); Osaka University, Osaka (JP)

(72) Inventors: Atsunari Kawashima, Osaka (JP); Norio Nonomura, Osaka (JP); Ryoichi Imamura, Osaka (JP); Akinaru Yamamoto, Osaka (JP); Tomonori Kimura, Osaka (JP); Masashi Mita, Osaka (JP)

(73) Assignees: Kagami Inc., Osaka (JP); National Institutes of Biomedical Innovation, Health and Nutrition, Osaka (JP); Osaka University, Osaka (JP)

(21) Appl. No.: 18/598,428

(22) Filed: Mar. 7, 2024

(30) Foreign Application Priority Data
Mar. 8, 2023 (JP) 2023-036041

(51) Int. Cl. G16H 50/20; G01N 33/68; G01N 33/70; G16H 20/10

(52) U.S. Cl. CPC G16H 50/20; G01N 33/6812; G01N 33/6893; G01N 33/70; G16H 20/10; G01N 2333/76; G01N 2333/8139; G01N 2800/347

(57) ABSTRACT

Provided is a method for further improving therapeutic effect and cost efficiency by ascertaining cancer type and treatment responsiveness for individual patients. The invention provides a method which includes providing information regarding cancer type of a subject, comprising assessing the cancer type of the subject using a marker relating to the amount of a D-amino acid in blood of the subject, and providing information relating to the cancer type of the subject, based on the assessment results, as well as a system for providing information pertaining to cancer in a subject, which carries out the method.

### FIELD

The present invention relates to a method for providing information pertaining to cancer type and to a system for providing information pertaining to cancer type.

### BACKGROUND

Cells that proliferate autonomously without normal regulatory control due to a genetic abnormality in the cells are collectively referred to as a tumor, and when they have undergone invasive cell proliferation and metastasis. they are referred to as cancer (malignant tumor). Cancer treatment methods include surgery, radiotherapy, chemotherapy, drug therapy and immunotherapy, which are carried out either alone or in appropriately selected combinations as multidisciplinary treatment. As a result of progress in cancer research since the late 1990s it has become clear that drug response in chemotherapy and drug therapy, including both effects and side-effects, differs depending on the nature of the cancer, and therefore markers (biomarkers) have been developed for selection of optimal drugs. Biomarkers include those used to confirm individual features or types for determining changes in proteins or genes in blood, urine, saliva, cells and tissues, and in tumor markers such as used in health examination or complete medical checkups, such as PSA for prostate cancer. Current biomarkers in use include EGFR gene mutations and ALK fused gene in lung cancer, as well as HER2 protein overexpression in breast cancer or stomach cancer, in connection with the action mechanisms of drugs which target molecules associated with tumor proliferation (molecular targeted drugs). For example, EGFR gene mutation markers are used to predict the therapeutic effects of EGFR tyrosine kinase inhibitors (EGFR inhibitors). Immune checkpoint inhibitors are also being researched as candidate markers, including microsatellite instability, tumor mutation burden, PD-L1 positivity rate and Epstein-Barr virus. Urothelial carcinoma markers such as BTA, NMP22, ImmnoCyt and UroVysion are used in the clinic, but they all have low sensitivity and low specificity while being costly, and thus fail to meet needs in the clinic.

Recent advances in quantitative research on distinguishing trace D-amino acids and L-amino acids in vivo including mammals, owing to higher-level performance of techniques used to identify and analyze chiral amino acids, has led to elucidation of the presence and function of some D-amino acids which were previously treated as total amino acids (D-amino acids+L-amino acids), or as L-amino acids for convenience, due to technical limitations of the prior art. It has been reported that D-amino acids are present in varying levels in vivo, tissues, cells and body fluids depending on effects such as intake, symbiotic bacteria, metabolism (decomposition and synthesis), transport and excretion (PTL 3 and NPLs 1 to 5), that a characteristic chiral amino acid profile is exhibited in diseases such as kidney disease and other physical conditions (PTL 1), that D-amino acids are involved in intestinal immunity (NPL 6) and protect kidney-derived cells (NPL 2), and that carbohydrate metabolism in neurons is involved in D-serine biosynthesis (NPL 7). It has been disclosed that cancer patient blood shows fluctuations in D-serine, D-threonine, D-alanine, D-asparagine, D-allothreonine, D-glutamine, D-proline and D-phenylalanine in kidney cancer, D-histidine and D-asparagine in prostate cancer and D-alanine in lung cancer (PTL 1). However, there has been no evaluation of profiles among cancer types based on the amount of D-amino acids.

### CITATION LIST

### Patent Literature

[PTL 1] International Patent Publication No. WO2013/140785
[PTL 2] Japanese Patent Publication No. 4857071
[PTL 3] International Patent Publication No. WO2020/196436

### Non Patent Literature

[NPL 1] Y. Miyoshi, R. Konno, J. Sasabe, K. Ueno, Y. Tojo, M. Mita, S. Aiso and K. Hamase, Alteration of intrinsic amounts of D-serine in the mice lacking serine racemase and D-amino acid oxidase, Amino Acids, 43,1919-1931 (2012). DOI: 10.1007/s00726-012-1398-4
[NPL 2] Y. Nakade, Y. Iwata, K. Furuichi, M. Mita, K. Hamase, R. Konno, T. Miyake, N. Sakai, S. Kitajima, T. Toyama, Y. Shinozaki, A. Sagara, T. Miyagawa, A. Hara, M. Shimizu, Y. Kamikawa, K. Sato, M. Oshima, S. Yoneda-Nakagawa, Y. Yamamura, S. Kaneko, T. Miyamoto, M. Katane, H. Homma, H. Morita, W. Suda, M. Hattori and T. Wada, Gut microbiota-derived D-serine protects against acute kidney injury, JCI Insight, 3 (20) (2018). DOI: 10.1172/jci.insight.97957
[NPL 3] M. Ariyoshi, M. Katane, K. Hamase, Y. Miyoshi, M. Nakane, A. Hoshino, Y. Okawa, Y. Mita, S. Kaimoto, M. Uchihashi, K. Fukai, K. Ono, S. Tateishi, D. Hato, R. Yamanaka, S. Honda, Y. Fushimura, E. Iwai-Kanai, N. Ishihara, M. Mita, H. Homma and S. Matoba, D-Glutamate is metabolized in the heart mitochondria, Scientific Reports, 7,43911 (2017). DOI: 10.1038/srep43911
[NPL 4] P. Wiriyasermkul, S. Moriyama, Y. Tanaka, P. Kongpracha, N. Nakamae, M. Suzuki, T. Kimura, M. Mita, J. Sasabe and S. Nagamori, D-Serine, an emerging biomarker of kidney diseases, is a hidden substrate of sodium-coupled monocarboxylate transporters, bioRxiv preprint. DOI: 10.1101/2020.08.10.244822
[NPL 5] A. Hesaka, S. Sakai, K. Hamase, T. Ikeda, R. Matsui, M. Mita, M. Horio, Y. Isaka and T. Kimura, D-Serine reflects kidney function and diseases, Scientific Reports, 9,5104 (2019). DOI: 10.1038/s41598-019-41608-0
[NPL 6] J. Sasabe, Y. Miyoshi, S. Rakoff-Nahoum, T. Zhang, M. Mita, B. M. Davis, K. Hamase and M. K. Waldor, Interplay between microbial D-amino acids and host D-amino acid oxidase modifies murine mucosal defence and gut microbiota, Nature Microbiology, 1,16125 (2016). DOI: 10.1038/nmicrobiol.2016.125
[NPL 7] M. Suzuki, J. Sasabe, Y. Miyoshi, K. Kuwasako, Y. Muto, K. Hamase, M. Matsuoka, N. Imanishi and S. Aiso, Glycolytic flux controls D-serine synthesis through glyceraldehyde-3-phosphate dehydrogenase in astrocytes, Proceedings of the National Academy of Sciences of the United States of America, 112 (17), E2217-E2224 (2015). DOI: 10.1073/pnas.1416117112
[NPL 8] N. Okamoto, Y. Miyagi, A. Chiba, M. Akaike, M. Shiozawa, A. Imaizumi, H. Yamamoto, T. Ando, M. Yamakado and O. Tochikubo, Diagnostic modeling with differences in plasma amino acid profiles between non-cachectic colorectal/breast cancer patients and healthy individuals, International Journal of Medicine and Medical Sciences, 1 (1), 001-008 (2009). DOI: 10.5897/IJMMS.90,00074
[NPL 9] Okamoto, N., “Cancer screening using AminoIndex(registered trademark) technology”, Ningen Dock, 26 (3), 454-466 (2011). DOI:10.11320/ningendock.26.454

### SUMMARY

### Technical Problem

Cancer is the number one cause of death, and while many treatment alternatives exist including surgery, radiotherapy, chemotherapy, drug therapy and immunotherapy, it is still desirable to develop improved methods with greater therapeutic effects and better cost efficiency, based on cancer type and treatment responsiveness for individual patients.

### Solution to Problem

The present inventors have comprehensively quantified and analyzed chiral amino acids (D-amino acids and L-amino acids) in cancer patient blood, and have found chiral amino acid levels in blood to be correlated with cancer type and treatment responsiveness. As a result of detailed research in this regard, it was found that developing markers based on the amount of the D-amino acids is clinically useful for assessing cancer type and treatment responsiveness in the clinic, and the present invention was developed to provide a feasible solution method. Specifically, the scope of the invention encompasses the following.

[1] A method for providing information regarding cancer type of a subject, comprising:
assessing the cancer type of the subject using a marker relating to the amount of a D-amino acid in blood of the subject; and
providing information relating to the cancer type of the subject, based on the assessment results.

[2] The method according to [1] above, wherein:
the marker relating to the amount of a D-amino acid in blood of the subject is used to assess the cancer type and treatment responsiveness of the subject, and
information relating to the cancer type and treatment responsiveness of the subject is provided, based on the assessment results.

[3] The method according to [1] or [2] above, wherein the marker relating to the amount of a D-amino acid is a formula or value obtained by calibrating the amount of the D-amino acid with an in vivo substance of the subject.

[4] The method according to [3] above, wherein the in vivo substance is an L-amino acid.

[5] The method according to [1] or [2] above, wherein the marker relating to the amount of a D-amino acid is a formula or value obtained by calibrating the amount of the D-amino acid with a marker relating to kidney function in the subject.

[6] The method according to [5] above, wherein the marker relating to kidney function is the amount of one or more factors selected from the group consisting of creatinine, cystatin C, inulin clearance, creatinine clearance, D-amino acid clearance, urine protein, urine albumin, β2-MG, α1-MG, NAG, L-FABP and NGAL.

[7] The method according to any one of [1] to [6] above, wherein the D-amino acid is one or more selected from the group consisting of D-proline, D-serine, D-alanine and D-asparagine.

[8] The method according to any one of [1] to [7] above, wherein the cancer is urogenital cancer.

[9] The method according to [8] above, wherein the urogenital cancer is cancer selected from the group consisting of renal cell carcinoma, renal pelvis/ureter cancer, bladder cancer and prostate cancer.

[10] The method according to [8] above, wherein the urogenital cancer is cancer selected from the group consisting of clear cell carcinoma, urothelial carcinoma, adenocarcinoma and seminoma.

[11] The method according to any one of [1] to [10] above, wherein the assessment is made by comparing the marker relating to the amount of a D-amino acid with an assessment value for cancer type determined from the amount of the D-amino acid in the blood of subjects with cancer.

[12] A system for carrying out the method according to any one of [1] to [11] above, the system including a memory unit, an input unit, an analytical measurement unit, a data processing unit and an output unit, and the system being configured so that:
the input unit inputs information from a subject,
the analytical measurement unit acquires a marker relating to the amount of a D-amino acid of the subject by analytical measurement of information from the subject, inputted through the input unit,
the memory unit stores an assessment value related to cancer type,
the data processing unit assesses the cancer type of the subject by comparing the marker of the subject acquired by the analytical measurement unit, with the assessment value stored in the memory unit, and
the output unit outputs the assessment results from the data processing unit as information relating to the cancer type of the subject.

[13] The system according to [12] above, which is further configured so that:
the memory unit stores an assessment value relating to cancer type and treatment responsiveness,
the data processing unit assesses the cancer type and treatment responsiveness of the subject by comparing the marker of the subject acquired by the analytical measurement unit with the assessment value stored in the memory unit, and
the output unit outputs the assessment results from the data processing unit as information relating to the cancer type and treatment responsiveness of the subject.

### Advantageous Effects of Invention

According to the invention it is possible to use a marker relating to the amount of the D-amino acids of a subject with cancer to assess cancer type, or to analyze, extract, study, select or provide a suitable method of treatment on an individual level, thereby helping to control effects and side-effects, so as to provide precision medicine for improved patient QOL, and to lower medical costs.

### BRIEF DESCRIPTION OF DRAWINGS

FIG. 1 is a pair of graphs showing the amounts of D-amino acids in blood harvested from a healthy subject and a subject with urogenital cancer (test and validation cohorts).

FIG. 2-1 is a set of graphs showing ROC curves (Youden marker: ≥1.5) for discriminating subject cancer type (urothelial carcinoma vs. prostate cancer), using the amounts of D-amino acids and the D/L ratio between amino acids in blood as the explanatory variable.

FIG. 2-2 Same as above.

FIG. 2-3 Same as above.

FIG. 2-4 Same as above.

FIG. 2-5 Same as above.

FIG. 2-6 Same as above.

FIG. 2-7 Same as above.

FIG. 2-8 Same as above.

FIG. 3-1 is a set of graphs showing ROC curves (Youden marker: ≥1.5) for discriminating subject cancer type (urothelial carcinoma vs. renal cell carcinoma), using the amounts of D-amino acids and the D/L ratio between amino acids in blood as the explanatory variable.

FIG. 3-2 Same as above.

FIG. 3-3 Same as above.

FIG. 3-4 Same as above.

FIG. 3-5 Same as above.

FIG. 3-6 Same as above.

FIG. 3-7 Same as above.

FIG. 3-8 Same as above.

FIG. 4-1 is a set of graphs showing ROC curves (Youden marker: ≥1.5) for discriminating subject cancer type (renal cell carcinoma vs. prostate cancer), using the amounts of D-amino acids and the D/L ratio between amino acids in blood as the explanatory variable.

FIG. 4-2 Same as above.

FIG. 4-3 Same as above.

FIG. 4-4 Same as above.

FIG. 4-5 Same as above.

FIG. 4-6 Same as above.

FIG. 4-7 Same as above.

FIG. 4-8 Same as above.

FIG. 4-9 Same as above.

FIG. 4-10 Same as above.

FIG. 4-11 Same as above.

FIG. 4-12 Same as above.

FIG. 5 is a pair of survival curves for progression-free survival periods and cancer disease specific survival periods with administration of an anti-PD-1 antibody formulation to subjects with urogenital cancer, in a low plasma D-alanine level group and a high plasma D-alanine level group.

FIG. 6 is a block diagram showing an example of the configuration of the system of the invention.

FIG. 7 is a flow chart schematically showing an example of treatment (invention method) using the system of the invention.

### DESCRIPTION OF EMBODIMENTS

Embodiments for carrying out the invention will be described in detail below, with the understanding that the technical scope of the invention is not limited only to these embodiments. The prior art documents cited throughout are incorporated herein by reference.

Throughout the present specification, the amino acids and bound amino acid residues may be represented by their three-letter abbreviations known to those skilled in the art. The major ones used herein are as follows.

TABLE 1

| Abbreviation | Definition |
|---|---|
| Ala | Alanine |
| Arg | Arginine |
| Asn | Asparagine |
| Asp | Aspartic acid |
| Cys | Cysteine |
| Gln | Glutamine |
| Glu | Glutamic acid |
| Gly | Glycine |
| His | Histidine |
| Ile | Isoleucine |
| Leu | Leucine |
| Lys | Lysine |
| Met | Methionine |
| Phe | Phenylalanine |
| Pro | Proline |
| Ser | Serine |
| Thr | Threonine |
| Trp | Tryptophan |
| Tyr | Tyrosine |
| Val | Valine |
| D-AA | D-Amino acid |
| L-AA | L-Amino acid |

The present invention provides, as a novel evaluation approach for cancer, a method of improving diagnosis precision and assisting selection of appropriate treatment means using a marker relating to the amount of a D-amino acid in blood.

According to one embodiment, the invention can provide a method for providing information relating to cancer type of a subject using a marker relating to the amount of a D-amino acid in the blood of the subject. According to another embodiment, the invention can provide a method of providing information relating to cancer type and treatment responsiveness of a subject using a marker relating to the amount of a D-amino acid in the blood of the subject. Specifically, the invention according to one embodiment provides a method for providing information pertaining to cancer of a subject using a marker relating to the amount of a D-amino acid in the blood of the subject, wherein the information is selected from the group consisting of:
the cancer type of the subject; and
the treatment responsiveness of the subject.

Throughout the present specification, the terms “cancer” and “cancer type” are not particularly restricted and include, for example, leukemia (such as acute myelocytic leukemia, chronic myelogenous leukemia, acute lymphocytic leukemia and chronic lymphatic leukemia), malignant lymphoma (Hodgkin's lymphoma, non-Hodgkin lymphoma (such as adult T cell leukemia, follicular lymphoma and diffuse large B-cell lymphoma)), multiple myeloma, myelodysplastic syndrome, head and neck cancer, gastrointestinal cancer (such as esophageal cancer, esophageal adenocarcinoma, stomach cancer, colorectal cancer, colon cancer and rectum cancer), liver cancer (such as hepatocellular carcinoma), gallbladder/cholangiocarcinoma, biliary tract cancer, pancreatic cancer, thyroid cancer, lung cancer (such as non-small-cell lung carcinoma (including squamous epithelium non-small-cell lung carcinoma and non-squamous non-small cell lung carcinoma) and small-cell lung cancer), breast cancer, ovarian cancer (such as serous ovarian cancer), cervical cancer, uterine cancer, endometrial cancer, vaginal cancer, vulvar cancer, renal carcinoma (such as renal cell carcinoma), urothelial carcinoma (such as bladder cancer and upper urinary tract cancer), prostate cancer, testicular cancer (such as germ cell tumor), bone/soft tissue sarcoma, skin cancer (such as uveal melanoma, malignant melanoma and Merkel cell carcinoma), glioma, brain tumor (such as glioblastoma), pleural mesothelioma and cancer of unknown origin, any of which may be malignant tumors or benign tumors. The type of cancer for which the invention is applied is preferably urogenital cancer. Without being limitative, the invention can be used to discriminate whether a subject suffers from urogenital cancer, and for example, it can discriminate between urogenital cancer selected from the group consisting of renal cell carcinoma, renal pelvis and ureter cancer, bladder cancer and prostate cancer, as well as urogenital cancer selected from the group consisting of clear cell carcinoma, urothelial carcinoma, adenocarcinoma and seminoma.

The term “D-amino acids” is used herein to include amino acids that are constituents of “D-form” proteins, as stereoisomers of amino acids that are constituents of “L-form” proteins, as well as glycine which has no stereoisomer, and specifically they include glycine, D-alanine, D-histidine, D-isoleucine, D-alloisoleucine, D-leucine, D-lysine, D-methionine, D-phenylalanine, D-threonine, D-allothreonine, D-tryptophan, D-valine, D-arginine, D-cysteine, D-glutamine, D-proline, D-tyrosine, D-aspartic acid, D-asparagine, D-glutamic acid and D-serine. Since D-cysteine in a biological sample is oxidized ex vivo and converted to D-cystine, one embodiment of the invention allows measurement of D-cystine instead of D-cysteine to determine the amount of D-cysteine in the biological sample.

The “amount of D-amino acid in blood” referred to herein may be the amount of the D-amino acid in a specified amount of blood volume, or it may the concentration. The amount of D-amino acid in blood is measured as the amount in a sample of blood that has been treated by centrifugal separation, sedimentation separation or other pretreatment for analysis. Therefore, the amount of the D-amino acid in blood can be measured as the amount in a blood sample derived from sampled whole blood, serum or blood plasma. For analysis using HPLC, as one example, the amount of the D-amino acid in a predetermined amount of blood may be represented in a chromatogram, and the peak heights, areas and shapes may be quantified by analysis based on standard sample comparison and calibration.

The amount of a D-amino acid and/or L-amino acid may be measured by any method, such as chiral column chromatography, or measurement using an enzyme method, or quantitation by an immunological method using a monoclonal antibody that distinguishes between optical isomers of amino acids. Measurement of the amount of a D-amino acid and/or L-amino acid in a sample according to the invention may be carried out using any method well known to those skilled in the art. Examples include chromatographic and enzyme methods (Y. Nagata et al., Clinical Science, 73 (1987), 105. Analytical Biochemistry, 150 (1985), 238., A. D'Aniello et al., Comparative Biochemistry and Physiology Part B, 66 (1980), 319. Journal of Neurochemistry, 29 (1977), 1053., A. Berneman et al., Journal of Microbial & Biochemical Technology, 2 (2010), 139., W. G. Gutheil et al., Analytical Biochemistry, 287 (2000), 196., G. Molla et al., Methods in Molecular Biology, 794 (2012), 273., T. Ito et al., Analytical Biochemistry, 371 (2007), 167.), antibody methods (T. Ohgusu et al., Analytical Biochemistry, 357 (2006), 15), gas chromatography (GC) (H. Hasegawa et al., Journal of Mass Spectrometry, 46 (2011), 502., M. C. Waldhier et al., Analytical and Bioanalytical Chemistry, 394 (2009), 695., A. Hashimoto, T. Nishikawa et al., FEBS Letters, 296 (1992), 33., H. Bruckner and A. Schieber, Biomedical Chromatography, 15 (2001), 166., M. Junge et al., Chirality, 19 (2007), 228., M. C. Waldhier et al., Journal of Chromatography A, 1218 (2011), 4537), capillary electrophoresis methods (CE) (H. Miao et al., Analytical Chemistry, 77 (2005), 7190., D. L. Kirschner et al., Analytical Chemistry, 79 (2007), 736., F. Kitagawa, K Otsuka, Journal of Chromatography B, 879 (2011), 3078., G. Thorsen and J. Bergquist, Journal of Chromatography B, 745 (2000), 389), and high-performance liquid chromatography (HPLC) HPLC) (N. Nimura and T. Kinoshita, Journal of Chromatography, 352 (1986), 169., A. Hashimoto et al., Journal of Chromatography, 582 (1992), 41., H. Bruckner et al., Journal of Chromatography A, 666 (1994), 259., N. Nimura et al., Analytical Biochemistry, 315 (2003), 262., C. Muller et al., Journal of Chromatography A, 1324 (2014), 109., S. Einarsson et al., Analytical Chemistry, 59 (1987), 1191., E. Okuma and H. Abe, Journal of Chromatography B, 660 (1994), 243., Y. Gogami et al., Journal of Chromatography B, 879 (2011), 3259., Y. Nagata et al., Journal of Chromatography, 575 (1992), 147., S. A. Fuchs et al., Clinical Chemistry, 54 (2008), 1443., D. Gordes et al., Amino Acids, 40 (2011), 553., D. Jin et al., Analytical Biochemistry, 269 (1999), 124., J. Z. Min et al., Journal of Chromatography B, 879 (2011), 3220., T. Sakamoto et al., Analytical and Bioanalytical Chemistry, 408 (2016), 517., W. F. Visser et al., Journal of Chromatography A, 1218 (2011), 7130., Y. Xing et al., Analytical and Bioanalytical Chemistry, 408 (2016), 141., K. Imai et al., Biomedical Chromatography, 9 (1995), 106., T. Fukushima et al., Biomedical Chromatography, 9 (1995), 10., R. J. Reischl et al., Journal of Chromatography A, 1218 (2011), 8379., R. J. Reischl and W. Lindner, Journal of Chromatography A, 1269 (2012), 262., S. Karakawa et al., Journal of Pharmaceutical and Biomedical Analysis, 115 (2015), 123., Hamase K, et al., Chromatography 39 (2018) 147-152).

The separative analysis system for optical isomers to be applied for the invention may be a combination of multiple separative analysis methods. More specifically, the amount of a D-amino acid and/or L-amino acid in a sample can be measured using an optical isomer analysis method comprising a step of passing a sample containing a component with optical isomers through a first column filler as the stationary phase, together with a first liquid as the mobile phase. to separate the components in the sample, a step of separately holding each of the components in the sample in a multi loop unit, a step of passing each of the components in the sample that are separately held in the multi loop unit through a flow channel in a second column filler having an optically active center, as the stationary phase, together with a second liquid as the mobile phase, to separate the optical isomers among each of the sample components, and a step of detecting the optical isomers in each of the sample components (see Japanese Patent Publication No. 4291628, for example). In HPLC analysis, D- and L-amino acids are sometimes pre-derivatized with a fluorescent reagent such as o-phthalaldehyde (OPA) or 4-fluoro-7-nitro-2,1,3-benzoxadiazole (NBD-F), or diastereomerized using an agent such as N-tert-butyloxycarbonyl-L-cysteine (Boc-L-Cys) (see Hamase, K. and Zaitsu, K., Bunseki Kagaku, Vol. 53, 677-690(2004), for example). Alternatively, the D-amino acids and/or L-amino acids may be measured by an immunological method using a monoclonal antibody that distinguishes optical isomers of amino acids, such as a monoclonal antibody that specifically binds to a D-amino acid or L-amino acid. When the total of D-amino acids and L-amino acids is to be used as the marker it is not necessary to separate the D-amino acids and L-amino acids, in which case the amino acids can be analyzed without separating the D-amino acids and L-amino acids. Separation and quantitation may also be carried out in such cases using an enzyme method, antibody method, GC, CE or HPLC.

Throughout the present specification, the amounts of biomolecules such as D-amino acids, L-amino acids, creatinine and proteins, or drugs, may be expressed in any physical quantity that can be measured, which includes not only the simple mass, weight and amount of substance (mol), but also the mass, weight or amount of substance (mol) per tissue, cell, organ or molecular units or per volume or weight, or the mass, weight, amount of substance (mol), concentration, specific gravity or density in a fluid such as blood or urine.

As used herein, “marker relating to the amount of a D-amino acid” means the value of a measured amount of D-amino acid, or D-amino acid clearance, or D-amino acid excretion rate (PTL 3, NPL 5), or a formula or value corrected for purpose, using the amount of a D-amino acid as the explanatory variable, or a value calculated from a set formula. The value measured for a subject is the examination value of the indicator relating to the amount of a D-amino acid. According to one aspect of the invention, the amount of the D-amino acid in the blood may be calibrated using a physiological variable factor such as age, gender or BMI. When the dynamics of the D-amino acid are affected by kidney function, it may be calibrated using a kidney function marker. Without intending to be limitative, the kidney function marker may be one or more selected from among creatinine, cystatin C, inulin clearance, creatinine clearance, D-amino acid clearance, urine protein, urine albumin, β2-MG, α1-MG, NAG, L-FABP, NGAL, glomerular filtration rate and estimated glomerular filtration rate (eGFR), with ratio of the amount of D-amino acid/the amount of creatinine as a specific example. Since D-amino acids in vivo are known to fluctuate in neurodegenerative diseases (such as ALS) and autoimmune disease (such as multiple sclerosis) (PTLs 1 to 2), they can be calibrated by fluctuation factors and markers in different diseases.

The “assessment of cancer type” using a marker relating to the amount of the D-amino acid may be carried out using an assessment value for the marker (“reference range or “clinical assessment value”, according to the invention). The assessment value (reference range or clinical assessment value) used for the invention is generally set at a 95% interval around the center of an examination value distribution for a subject with a specific cancer, according to one embodiment, but any interval may be set according to the purpose. The assessment value has a diagnosis threshold, treatment threshold or preventive medicine threshold based on a reference for assessing prognosis in regard to diagnosis, discrimination, prevention or treatment for a specific cancer type. The threshold (cutoff value) may be set by a case control study, clinical medicine empirical rule, case series study, cohort study or expert consensus, using analysis data or results relating to predictability and assessability utilizing an ROC curve (Receiver Operating Characteristic curve), multivariate logistic regression model or Cox proportional hazard model. According to one embodiment, assessment results for discriminating the cancer type of a subject can be provided, for example, by comparing a marker relating to the amount of the D-amino acids in the blood of the subject in need of assessment, with an assessment value determined from the amount of the D-amino acids in blood of a target group having a specific cancer. Specifically, it is possible to obtain information regarding whether the subject suffers from cancer, whether the cancer is a specific cancer type, or whether the cancer is not a specific cancer type. Such a subject may be a subject without cancer, even if suspected of having cancer.

Without being limitative, the “assessment value” (also referred to as “reference range” or “clinical assessment value”) used to discriminate cancer type according to the invention may be derived by comparing a marker relating to the amount of a D-amino acid in blood, for a first target group having a specific cancer type requiring discrimination, and a second target group having a different cancer type than the first target group. Moreover, without being limitative, this allows discrimination of whether a subject suffers from urogenital cancer, and for example, it can discriminate between urogenital cancer selected from the group consisting of renal cell carcinoma, renal pelvis and ureter cancer, bladder cancer and prostate cancer, as well as urogenital cancer selected from the group consisting of clear cell carcinoma, urothelial carcinoma, adenocarcinoma and seminoma.

The method used to compare a marker relating to the amount of the D-amino acids in the blood of the subject with a predetermined assessment value is not particularly restricted. For example, the assessment value may be used as the upper limit for a marker relating to the amount of a D-amino acid in the blood, allowing assessment based on whether or not the marker relating to the amount of the D-amino acid in the blood of the subject is at or above the assessment value, or whether or not it exceeds the assessment value. Alternatively, the assessment value may be used as the lower limit for a marker relating to the amount of a D-amino acid in the blood, allowing assessment based on whether or not the marker relating to the amount of the D-amino acid in the blood of the subject is at or below the assessment value, or whether or not it falls short of the assessment reference value. When an assessment reference range is used as the assessment value, assessment may be made, based on whether the marker relating to the amount of the D-amino acid in the blood of the subject is within the assessment reference range, or above the assessment reference range or below the assessment reference range.

In the method of the invention, the assessment value may be a single assessment value or an assessment reference range, or multiple assessment values or assessment reference ranges may be used in combination, or 1, 2 or more assessment values and 1, 2 or more assessment reference ranges may be used in combination.

The term “treatment responsiveness” as used herein means the nature of symptoms or pathology of a patient that allows prediction and estimation of future progress and outlook (prognosis) of the disease or treatment. Treatment responsiveness includes reaction to a drug (effect and side-effects), functional age, degree of cancer cachexia and state of body fat, allowing prediction and estimation regarding functional prognosis of organs of the subject with cancer, life prognosis and estimated death, and tumor reduction, growth, metastasis or relapse, by variables (parameters) and units appropriate for each evaluation. Treatment responsiveness is generally described as being successful, good or high, depending on the degree of effect exhibited in line with the purpose of treatment, or as being unsuccessful, poor or low, in the case of a low effect in terms of the purpose of treatment, or failure to achieve the purpose, or an effect counter to the purpose (side-effect). Specifically, treatment responsiveness may be considered to be high when the survival period has been extended by treatment using an anticancer agent, or treatment responsiveness may be considered to be low when the survival period has not been extended or has been reduced. Depending on the assessed level of “treatment responsiveness”, it is possible to select optimal means for a subject diagnosed with a specific disease, such means being surgery, radiotherapy, chemotherapy, drug therapy, immunotherapy, alimentary therapy or exercise therapy, or other technologies (such as surgical techniques or administration methods), or to decide on priority or on additional preparation of the patient so as to optimize the treatment means. The criteria and purpose of employing such means may be curing of the disease, symptom reduction or elimination, halting or slowing of disease progression, prevention of the disease or symptoms, inhibiting aggravation of the underlying disease, avoiding or minimizing side-effects, or improving or maintaining cost effectiveness and QOL. According to one embodiment, the information relating to treatment responsiveness of a patient with cancer may be provided, for example, by comparing the marker with an assessment value determined based on the amount of the D-amino acids in blood from subjects with cancer for which information relating to treatment responsiveness has been provided.

According to the invention, cancer type can be assessed using a marker relating to the amount of a D-amino acid in the blood of a subject with diagnosis of cancer or suspected cancer based on a clinical test. For a marker relating to the amount of a D-amino acid, as one aspect, different D-amino acid and L-amino acid profiles in blood from subjects, based on cancer type can be utilized for assessment of cancer type by comparing an examination value for a subject with an assessment value for a previously established marker relating to the amount of a D-amino acid (either a reference range or a clinical assessment value). Tumor markers (such as AFP, hCG, LDH or PSA for urogenital cancer) are substances produced by cancer cells or substances produced by reaction of patient cells with tumors, and their examination can be used for diagnosis of tumors or assessment of relapse, metastasis and therapeutic effects, but one problem is that healthy subjects and benign conditions can also exhibit positivity. Since intake, absorption, transport, distribution, metabolism (synthesis and decomposition), excretion and function of D-amino acids in blood partially vary due to cancer, fluctuation of an examination value used as a marker relating to the amount of a D-amino acid in blood differs in principle from fluctuation in conventional tumor markers, making the amount of the D-amino acid in blood highly useful for assessment of cancer type. When cancer is suspected, based on symptoms of a subject but the cancer type cannot be assessed by a designated examination, a marker relating to the amount of a D-amino acid may be used to assess the type, such as renal cell carcinoma, renal pelvis/ureter cancer, bladder cancer, prostate cancer or testicular cancer. AminoIndex(registered trademark) examination (NPLs 8 to 9) is based on amino acid levels regardless of stereoisomerism, whereas gene-related examination is based on genotype, and therefore markers relating to the amounts of the D-amino acids which allow for examination of the phenotype of disease symptoms while distinguishing between D-amino acids and L-amino acids, have different features from these examinations and can exhibit an effect of also allowing assessment of true or false test results for cancer type.

According to a different aspect, assessment results provided by the invention can be utilized for screening of cancer type or pathological diagnosis. According to yet another aspect, the assessment results for cancer type may be used for screening of an effect, side-effect or secondary reaction in drug development, or assessment in a clinical trial or for an alternative endpoint. For assessment of cancer type, one or more D-amino acid types, L-amino acid types, calibration factors and markers may be used as a set of multiple markers to be used simultaneously for a panel examination. The subject sample used may be the same as for examination, harvested at a different timing in relation to the response and properties against cancer. The subject may be a mammal such as a human, or an animal in which cancer has been induced by cancer cell graft, genetic modification or drugs, or it may be an individual, cells, tissue or organoid prepared as a prescribed cancer model.

According to the invention, a marker relating to the amount of a D-amino acid in the blood of a subject can also be used to assess treatment responsiveness of the subject.

Since the present invention is carried out by comparing a specified assessment value (reference range or clinical assessment value) with an examination value for a subject, it is a preliminary diagnostic method or auxiliary diagnostic method intended to increase diagnosis precision for a physician, based on validation results, without judgment by a physician. The method may be conducted by a non-physician such as a clinical tester, health examiner or data processing technician, or by an analysis system or program.

According to one embodiment, the invention can provide information for assessing cancer type and treatment responsiveness based on a marker relating to the amount of a D-amino acid in the blood of a subject. The difference in profiles of D-amino acids and L-amino acids in blood of a subject, as response or prognosis of cancer after treatment, can be used to assist in selecting optimal means such as surgery, radiotherapy, chemotherapy, drug therapy, immunotherapy, alimentary therapy or exercise therapy, or determining priority for the same, depending on comparison of an assessment value (reference range or clinical assessment value) previously set for a marker relating to the amount of a D-amino acid with the examination value for the subject. Chemotherapy is treatment of cancer using an anticancer agent, which can provide a wider systemic effect compared to surgery or radiotherapy which have local effects. While surgical management (reduction surgery, routine surgery or expansion surgery) or endoscopic treatment are usually selected for early cancer and advanced cancer, chemotherapy is sometimes implemented in combination with surgery. Chemotherapy is implemented before surgery for the purpose of alleviating bleeding and burden on the body by shrinking the cancer, and after surgery for the purpose of inhibiting relapse, metastasis and proliferation of remaining cancer. Chemotherapy is also selected for unresectable cases, but according to another aspect, information can be provided to assist selection of optimal drugs or decision regarding prioritization, using a marker relating to the amount of a D-amino acid. As a specific example, the possibility of administering a drug may be determined by previously setting an assessment value (reference range or clinical diagnosis value) for a marker relating to the amount of a D-amino acid, associated with an effect or side-effect of the drug or its secondary reactions, and comparing it with an examination value for the subject. According to another aspect, it is possible to provide information for predicting and assessing an effect or side-effect or secondary reaction after drug administration, depending on a marker relating to the amount of a D-amino acid, or for assisting a decision on continuing or halting administration or determining the dose or timing of administration. Drugs to be used include, but are not limited to, antineoplastic agents such as antimetabolites (e.g. methotrexate, fluorouracil (5-FU), tegafur-gimeracil-oteracil potassium combination (S-1), gemcitabine hydrochloride (GEM), levofolinate calcium (l-LV), folinate calcium (LV), tegafur-uracil combination, capecitabine or trifluridine-tipiracil hydrochloride combination), platinum formulations (e.g. cisplatin (CDDP), oxaliplatin, miriplatin hydrate), anthracyclines (e.g. doxorubicin hydrochloride and epirubicin hydrochloride), topoisomerase inhibitors (e.g. etoposide, irinotecan hydrochloride hydrate), microtubule inhibitors (e.g. vinblastine hydrochloride, paclitaxel, docetaxel hydrate), alkylating agents (e.g. streptozocin), molecular targeted drugs (anti-VEGF antibody formulations: e.g. bevacizumab, anti-EGFR antibody formulations: e.g. cetuximab and panitumumab, anti-HER2 antibody formulations: e.g. trastuzumab, anti-VEGFR antibody formulations: e.g. ramucirumab, BCR/ABL inhibitors: e.g. imatinib mesylate, multikinase inhibitors: e.g. sunitinib malate, regorafenib hydrate, sorafenib tosylate and lenvatinib mesylate, EGFR inhibitors: e.g. erlotinib hydrochloride, VEGF inhibitors: e.g. aflibercept beta and mTOR inhibitors: e.g. everolimus and temsirolimus), angiogenesis inhibitors: e.g. sunitinib malate and sorafenib tosylate, folic acid antagonists: e.g. methotrexate, immune checkpoint inhibitors (immune checkpoint molecule inhibitors selected from the group consisting of CTLA-4, PD-1, PD-L1, PD-L2, LAG-3, TIM3, BTLA, B7H3, B7H4, 2B4, CD160, A2aR, KIR, VISTA and TIGIT, for example, anti-PD-1 antibodies (nivolumab and pembrolizumab)), antitumor antibiotics (e.g. mitomycin C and bleomycin), adrenocortical steroids (e.g. prednisolone and budesonide), non-specific immunostimulants (e.g. dry BCG/Connaught strain, Japanese strain), ethinylestradiol-based formulations (e.g. estrogen), steroidal antiandrogens (ovarian follicle/progesterone formulations: e.g. chlormadinone acetate and allylestrenol), nonsteroidal antiandrogens (e.g. bicalutamide and flutamide), 5α-reductase inhibitors (e.g. dutasteride), LHRH agonists (e.g. leuprorelin acetate and goserelin acetate), alkylating agent sodium esters (e.g. estramustine phosphate hydrate), interleukin-2 (e.g. teceleukin), interferon formulations (e.g. interferon alpha and interferon alpha 2b), and Chinese herbal medicines. Typical regimens include, for renal cell carcinoma: nivolumab/ipilimumab therapy, pembrolizumab/axitinib therapy, nivolumab/cabosantinib therapy, pembrolizumab/lenvatinib therapy and avelumab/axitinib therapy; for prostate cancer: docetaxel therapy and cabazitaxel therapy; for bladder cancer: doxorubicin therapy, immunobladder therapy, doxorubicin therapy, epirubicin therapy, cisplatin/epirubicin therapy, gemcitabine/cisplatin therapy and ddMVAC therapy; and for urothelial carcinoma: GEM.CBDCA therapy, enfortumab/vedotin therapy and avelumab therapy; with molecular targeted drugs also being used depending on genetic testing and protein testing results for a subject, or microtubule inhibitors, topoisomerase inhibitors and immune checkpoint inhibitors being used in combination depending on therapeutic effect, and one aspect of the invention provides information necessary for selection of these regimens. For example, when an examination value for a marker relating to the amount of a D-amino acid is in a range judged suitable for use of an immune checkpoint inhibitor, it is possible to provide information necessary for selection, prioritization and switching of administration timing of the immune checkpoint inhibitor, in the context of a treatment policy for an enhanced therapeutic effect.

Another aspect of the invention provides a system or program that carries out the method for providing information pertaining to cancer for a subject, as described above. For example, the invention provides a system configured so that:
the input unit inputs information from a subject,
the analytical measurement unit acquires a marker relating to the amount of a D-amino acid of the subject by analytical measurement of information from the subject, inputted through the input unit,
the memory unit stores an assessment value related to cancer type and/or treatment responsiveness,
the data processing unit assesses the cancer type and/or treatment responsiveness of the subject by processing the marker of the subject acquired by the analytical measurement unit, based on the assessment value stored in the memory unit, and
the output unit outputs the assessment results from the data processing unit as information relating to the cancer type and/or treatment responsiveness of the subject.

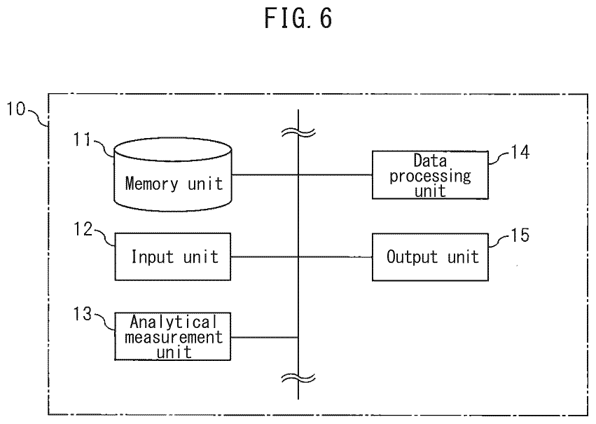

FIG. 6 is a block diagram schematically showing an example of a configuration of the system of the invention. However, the configuration shown in FIG. 6 is merely an example and is not intended to be limitative on the system of the invention. The sample analysis system 10 shown in FIG. 6 is constructed so as to allow the method of the invention to be carried out The sample analysis system 10 comprises a memory unit 11, an input unit 12, an analytical measurement unit 13, a data processing unit 14 and an output unit 15, and allows analysis of blood samples and output of information regarding cancer in a subject.

More specifically, the memory unit 11 in the sample analysis system 10 of the invention is configured so as to store various information including assessment values for cancer type and/or treatment responsiveness. The input unit 12 is configured to allow input of various information including data from a subject. The analytical measurement unit 13 is configured so as to carry out various analytical measurements such as acquiring a marker relating to the amount of a D-amino acid of a subject by analytical measurement of data from the subject. The data processing unit 14 is configured to be able to carry out various computation processing operations such as assessment of cancer type and/or treatment responsiveness of a subject, by processing a marker relating to the amount of a D-amino acid of the subject, based on the aforementioned assessment value. The output unit 15 is configured so as to be able to output various information including information relating to cancer type and/or treatment responsiveness.

The memory unit 11 has a portable storage device which may be a memory device such as a RAM, ROM or flash memory, a fixed disk device such as a hard disk drive, or a flexible disk or optical disk, for example. The memory unit 11 stores data measured by the analytical measurement unit 13, data and instructions inputted from the input unit 12, and results of computation processing by the data processing unit 14, as well as the computer program and database to be used for processing by the information processing equipment. The computer program may be a computer readable recording medium such as a CD-ROM or DVD-ROM, or it may be installed via the internet. The computer program is installed in the memory unit 11 using a commonly known setup program, for example. The memory unit 11 stores data for a cancer-related assessment value previously inputted through the input unit 12.

The input unit 12 also comprises an interface with units external to the sample analysis system 10, including operating units such as a keyboard and mouse. This allows the input unit 12 to input data measured by the analytical measurement unit 13 and instructions for computation processing to be carried out by the data processing unit 14. When the analytical measurement unit 13 is external, for example, the input unit 12 may also include an interface unit allowing input of measured data through a network or storage medium, separately from the operating device.

The analytical measurement unit 13 is configured so as to acquire markers relating to the amount of the D-amino acids of a subject by analytical measurement of data from the subject. For example, the analytical measurement unit 13 may configured to allow measurement of at least one amount of D-amino acid in a blood sample. The analytical measurement unit 13 may therefore have a construction allowing separation and measurement of the D-forms and L-forms of amino acids. The amino acids may be analyzed one type at a time, or some or all of the amino acid types may be analyzed at once. With no intention to be limitative, the analytical measurement unit 13 may be a chiral chromatography system comprising a sample introduction inlet, an optical resolution column and a detector, for example, and it is preferably a high-performance liquid chromatography system. From the viewpoint of detecting the levels of only specific amino acids, quantitation may be carried out by an enzyme method or immunological method. The analytical measurement unit 13 may be constructed separately from the sample analysis system 10, and measured data may be inputted through the input unit 12 using a network or storage medium.

The data processing unit 14 compares a marker relating to the amount of a D-amino acid with an assessment value stored in the memory unit, allowing selection of information regarding cancer of a subject (such as cancer type and/or treatment responsiveness). The marker relating to the amount of the D-amino acid may be a formula or value obtained by calibrating with the amount of an in vivo substance of the subject (for example, an L-amino acid level or a kidney function marker), or it may be a formula or value obtained by calibrating with a physiological variable factor such as age, gender or BMI.

The data processing unit 14 carries out various computation processing operations on the data measured by the analytical measurement unit 13 and stored in the memory unit 11, based on a program stored in the memory unit. The computation processing is carried out by a CPU in the data processing unit. The CPU includes a functional module that controls the analytical measurement unit 13, input unit 12, memory unit 11 and output unit 15, with the functional module performing various control operations. Each of the units may be constructed by independent integrated circuits, microprocessors and firmware.

The output unit 15 is constructed so as to output the information relating to cancer of the subject as the result of computation processing by the data processing unit. The output unit 15 may be output means such as a display device with a liquid crystal display that directly displays the computation processing results, or a printer, or it may be an interface unit for output to an external memory unit or output to a network.

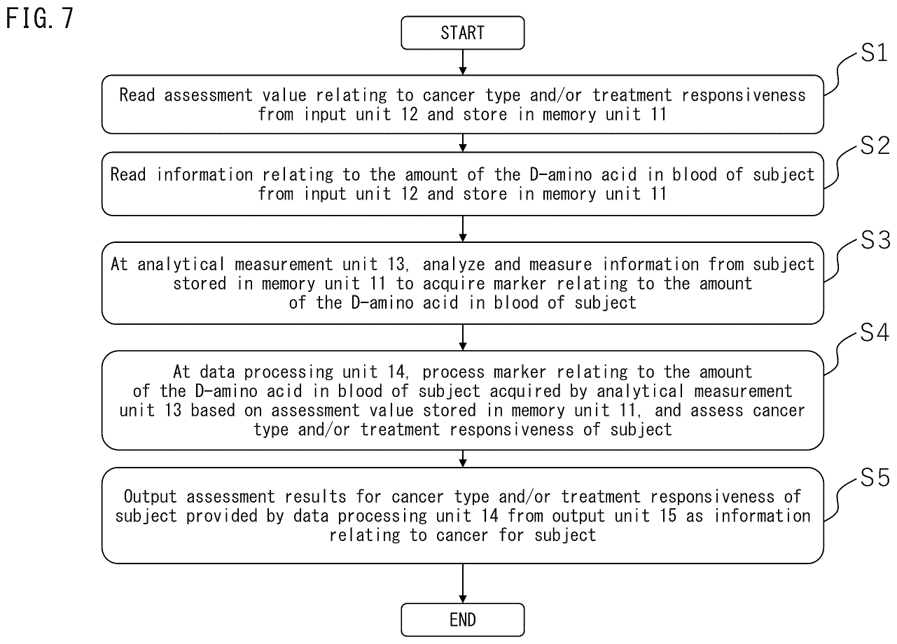

FIG. 7 is a flow chart schematically showing an example of treatment (invention method) using the system of the invention. However, the processing shown in FIG. 7 is merely an example and is not intended to be limitative on processing by the system of the invention. First, an assessment value relating to cancer type and/or treatment responsiveness is read into the input unit 12 and stored in the memory unit 11 (step S1). Next, information relating to the amount of a D-amino acid in the blood of the subject is read into the input unit 12 and stored in the memory unit 11 (step S2). At the analytical measurement unit 13, the information from the subject stored in the memory unit 11 is subjected to analytical measurement to acquire a marker relating to the amount of a D-amino acid in the blood of the subject (step S3). At the data processing unit 14, the marker relating to the amount of the D-amino acid in the blood of the subject acquired by the analytical measurement unit 13 is processed, based on the assessment value stored in the memory unit 11, and the cancer type and/or treatment responsiveness of the subject is assessed (step S4). Next, the assessment results for cancer type and/or treatment responsiveness of the subject provided by the data processing unit 14 are outputted from the output unit 15 as information relating to cancer for the subject (step S5).

According to another aspect, the invention may be a computer program that causes an information processor to carry out a method for providing information pertaining to cancer for the subject (hereunder referred to as “program of the invention” as appropriate). Specifically, the program of the invention may be configured as a program comprising computer instructions which, when installed in a general-purpose information processor, can cause the information processor or an external device such as an input/output interface or analyzer connected to it, to function as the sample analysis system 10 comprising the memory unit 11, input unit 12, analytical measurement unit 13, data processing unit 14 and output unit 15 shown in FIG. 6, for example. Such a program of the invention can be implemented using knowledge of computer programming common to those skilled in the art. The program of the invention and a recording medium such as a CD-ROM containing the program are also included within the technical scope of the invention.

According to one embodiment, the invention may be a method for treating cancer in which a subject is treated by cancer treatment means selected, based on information regarding cancer provided by an information device in which a method, system or program is installed. Referring to information regarding cancer provided by the invention allows optimal treatment means to be selected for the subject.

The content of all the patent and non-patent literature and references explicitly cited throughout the present specification are incorporated herein by reference.

The present invention will now be explained in detail by way of examples, with the understanding that these examples are in no way limitative of the invention. A person skilled in the art may easily implement modifications and changes to the invention, based on the description in the present specification, and these are also encompassed within the technical scope of the invention.

### EXAMPLES

The following symbols are used throughout the Examples.
D-AA: D-amino acid
L-AA: L-Amino acid
Asn: Asparagine
Ser: Serine
Ala: Alanine
Pro: Proline
PD-AA: Plasma D-amino acid concentration (nmol/mL)
PL-AA: Plasma L-amino acid concentration (nmol/mL)
PCre: Plasma creatinine concentration
PAAD/L: PD-AA/PL-AA

Subjects were either subjects with cancer diagnosed at Osaka University Hospital, or subjects without cancer, kidney or other disease diagnosis based on health examination, with plasma separated from blood collected from each subject after fasting for 2 hours or longer being provided for 2D-HPLC chiral amino acid analysis, and the acquired data being compared and analyzed. Both studies were approved by an ethics committee at Osaka University, with written informed consent being obtained from both participants.

Plasma taken from test subjects in a first cohort (test cohort) [renal cell carcinoma (n=7), urothelial carcinoma (n=11), prostate cancer (n=10), healthy subject (n=7)] and a second cohort (validation cohort) [urothelial carcinoma (n=92), healthy subject (n=60)] was analyzed for D-amino acids.

### (1) Discrimination of Cancer Using “PD-AA” as Marker

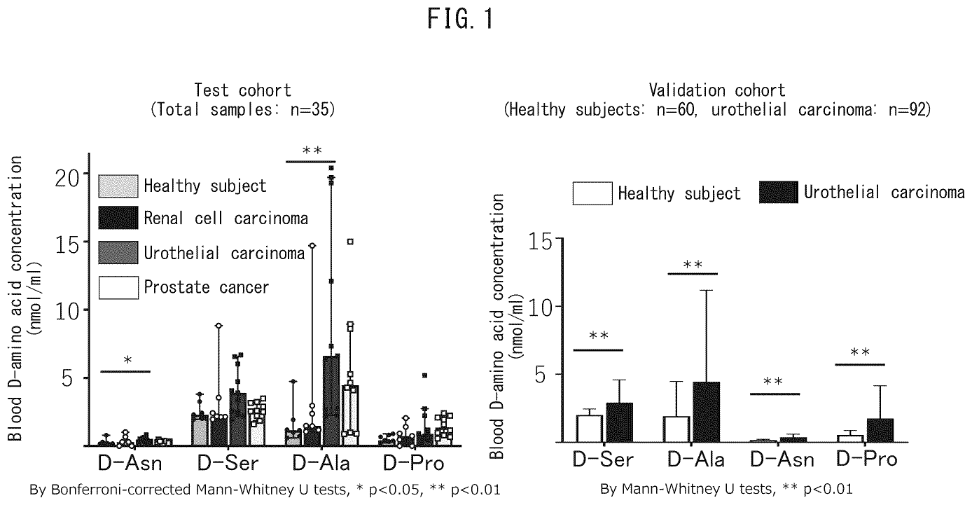

FIG. 1 shows PD-AA (nmol/mL) detected in subjects in each cancer type group.

The urothelial carcinoma group, and the target groups with cancer in all of the cohorts, showed increase in PD-Asn, PD-Ser, PD-Ala and PD-Pro compared to the healthy group, with increasing PD-AA in the order: urothelial carcinoma group, renal cell carcinoma group, prostate cancer group, allowing them to be discriminated. The PD-Asn and PD-Ala levels of the subjects with urothelial carcinoma showed the greatest increase compared to the other cancer types, allowing effective discriminatory assessment using one or more markers relating to the amounts of the D-amino acids in blood.

The predictability and discrimination ability for urothelial carcinoma by ROC (Receiver Operating Characteristic) curve analysis using PD-Asn resulted in AUC (Area under the curve)=0.857 in the first cohort and AUC=0.992 in the second cohort.

### (2) Discrimination of Cancer Type Using “PD-AA” and “PAAD/L” Markers

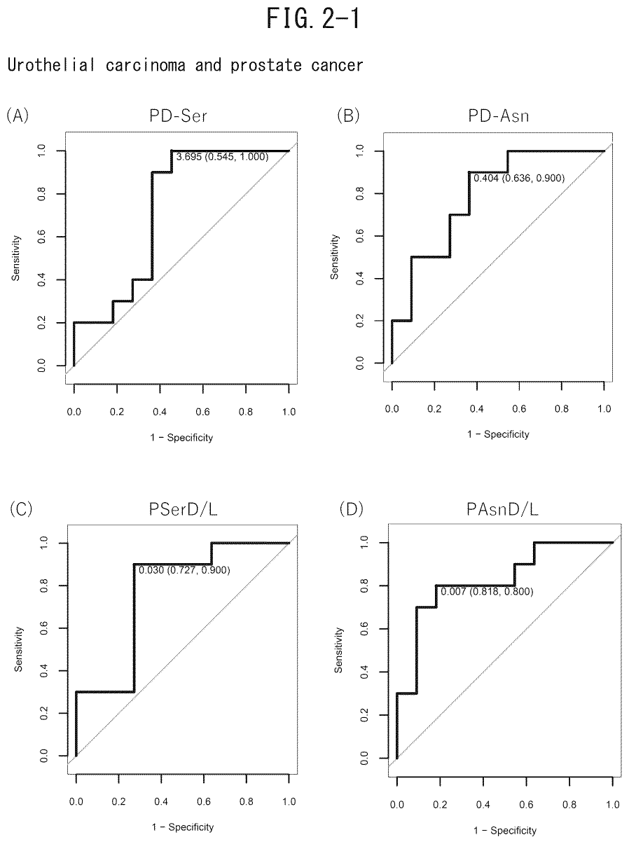

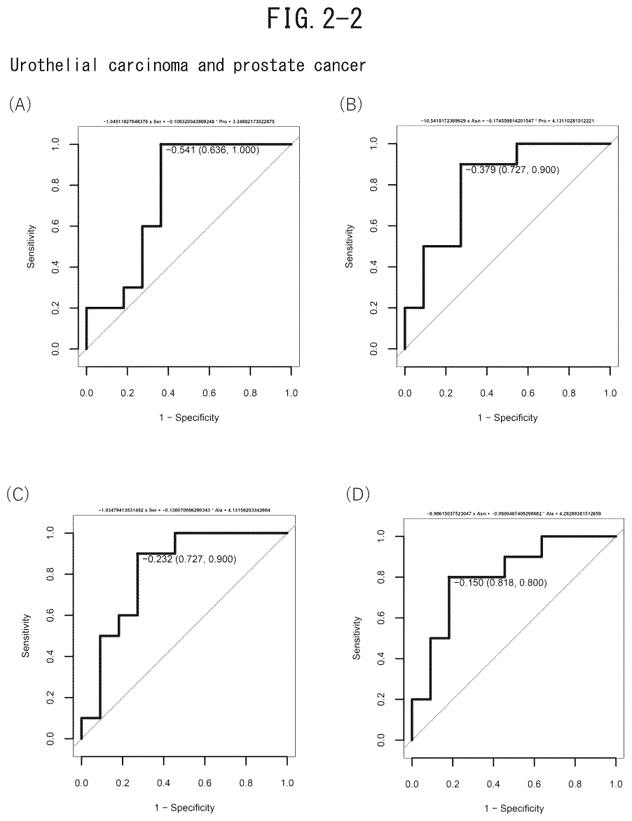

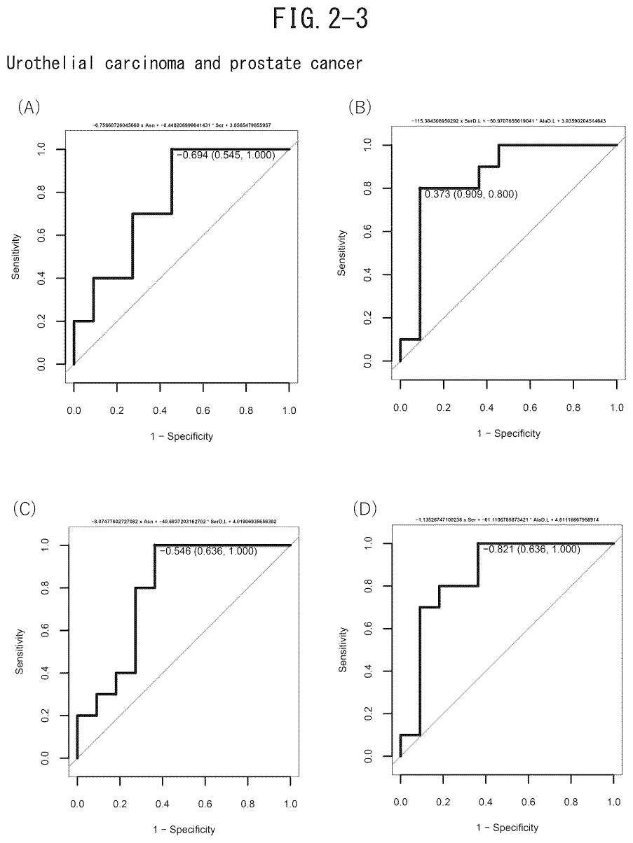

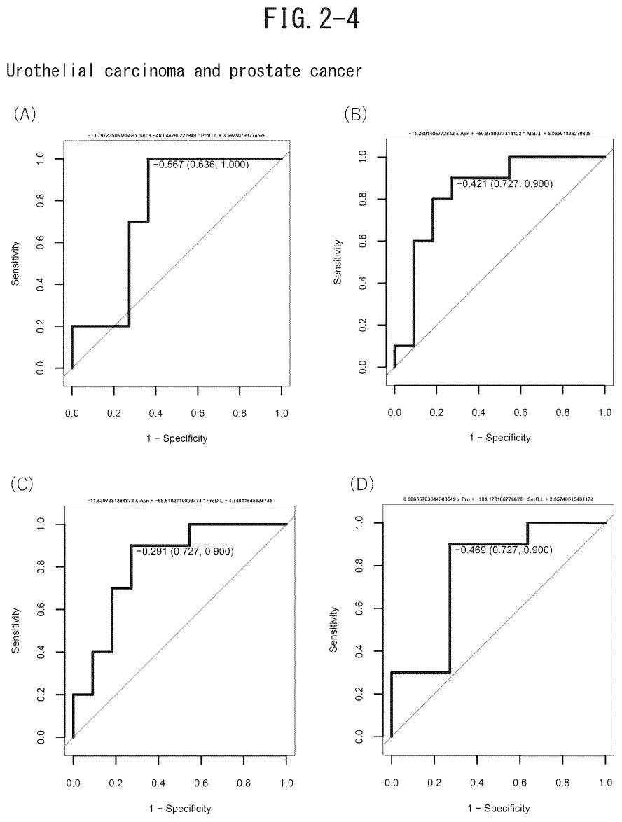

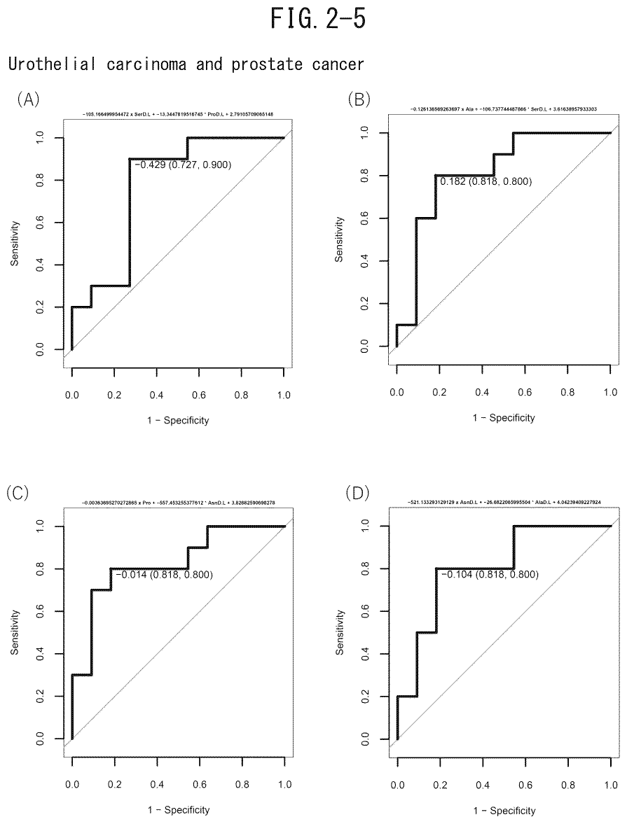

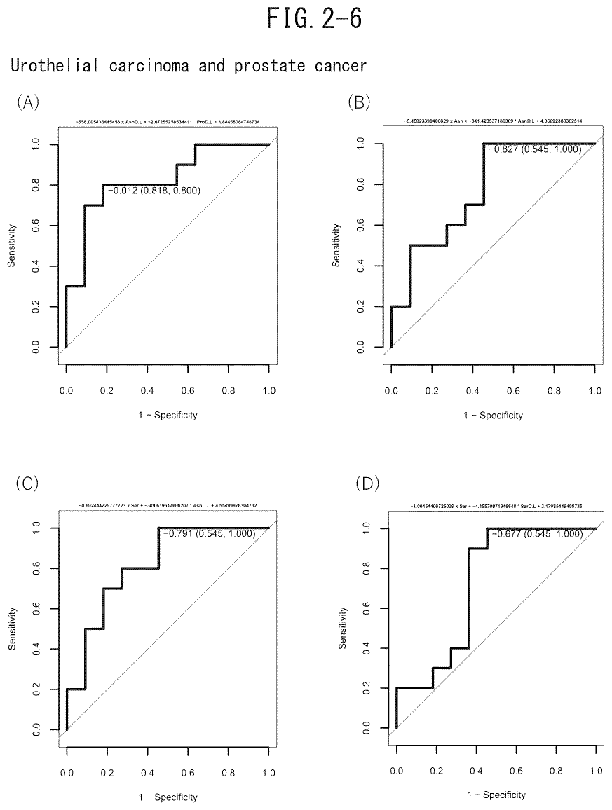

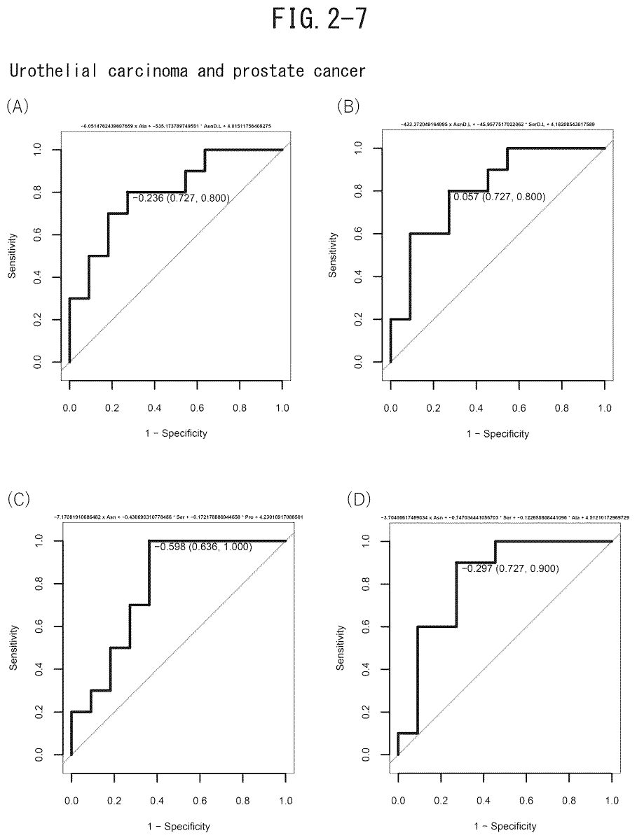

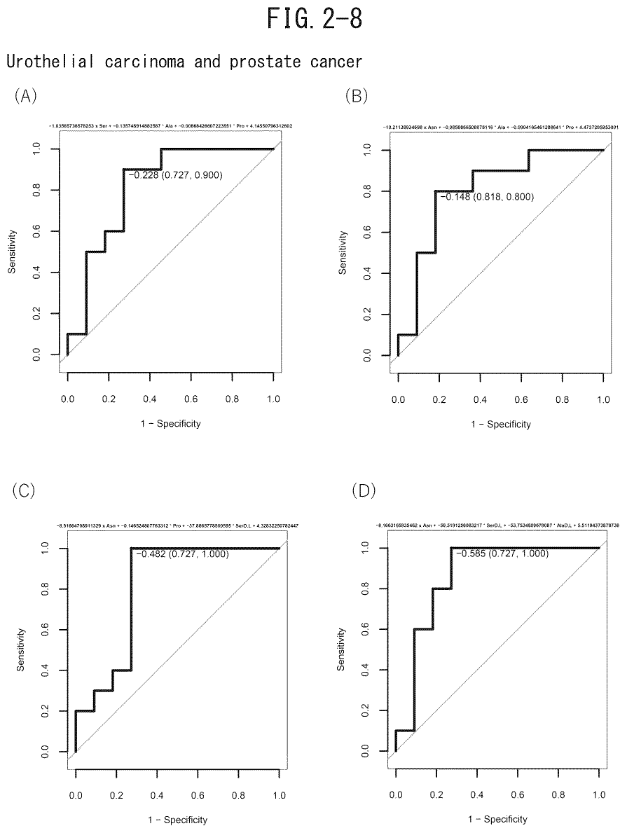

FIGS. 2 to 4 show ROC curves for prediction formulas using markers relating to the amounts of the D-amino acids as the explanatory variable, for the first cohort. The assessment values (cutoff values) for the ROC curves were derived from the points furthest from the ROC curve for the variable with the lowest predictive/diagnostic power, i.e. from the diagonal dotted line at AUC=0.500. Specifically, the point for the maximum calculated by (sensitivity+specificity−1) (Youden marker) was used as the cutoff value.

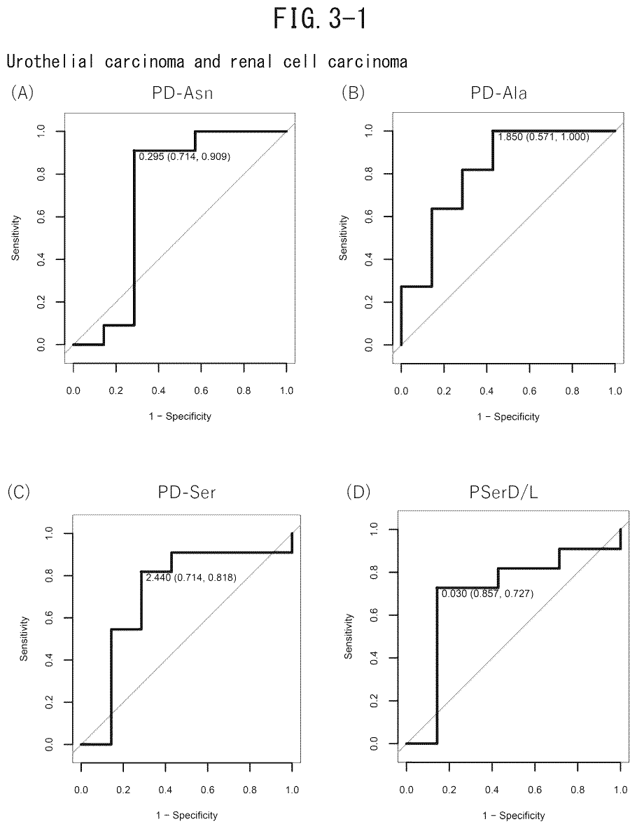

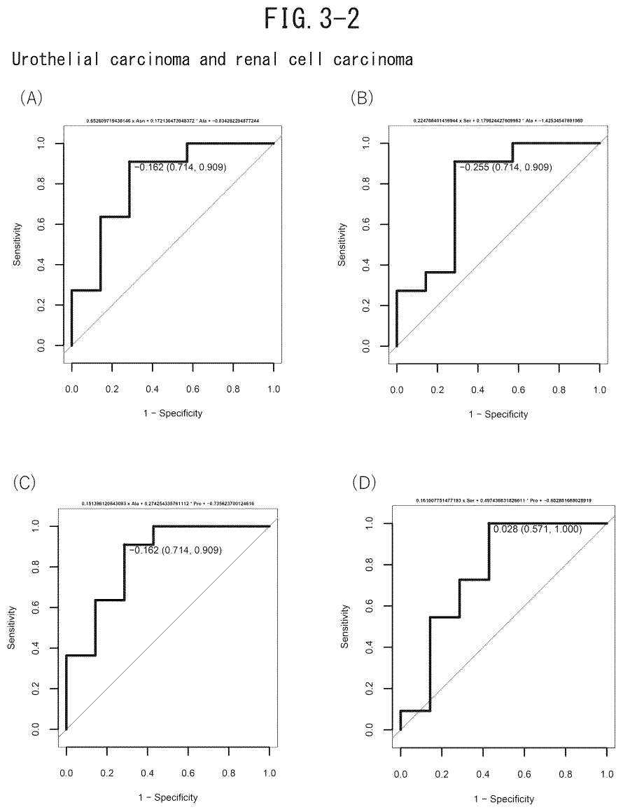

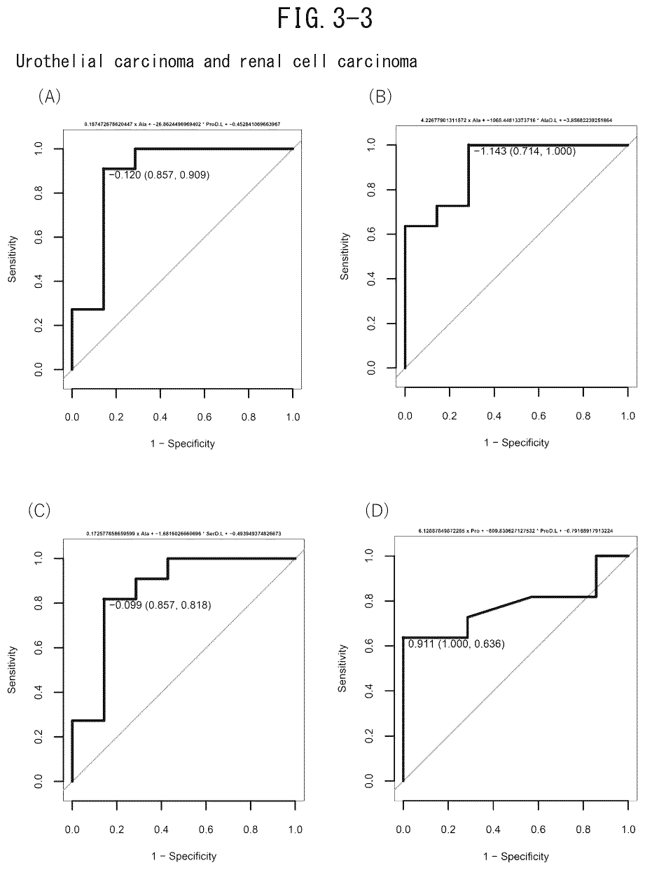

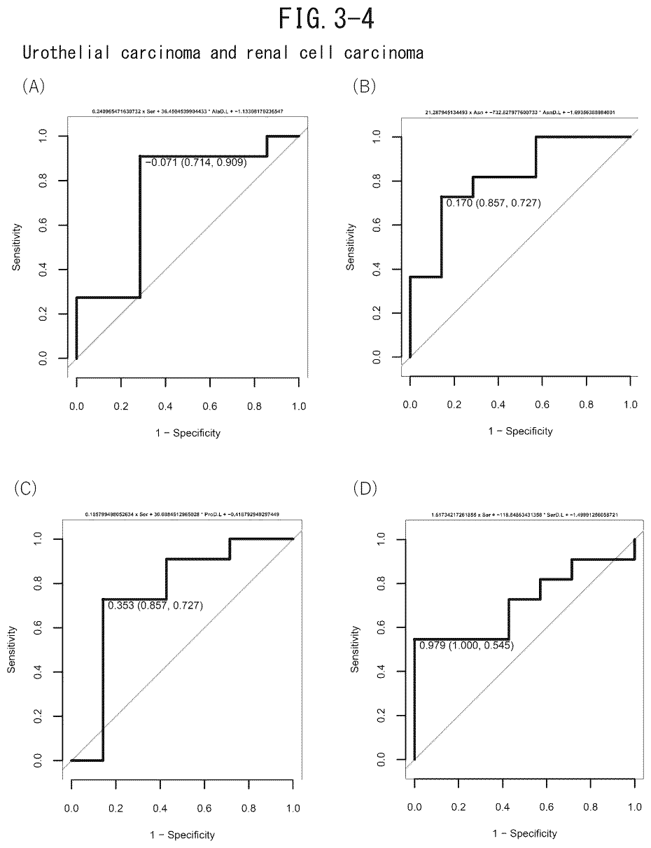

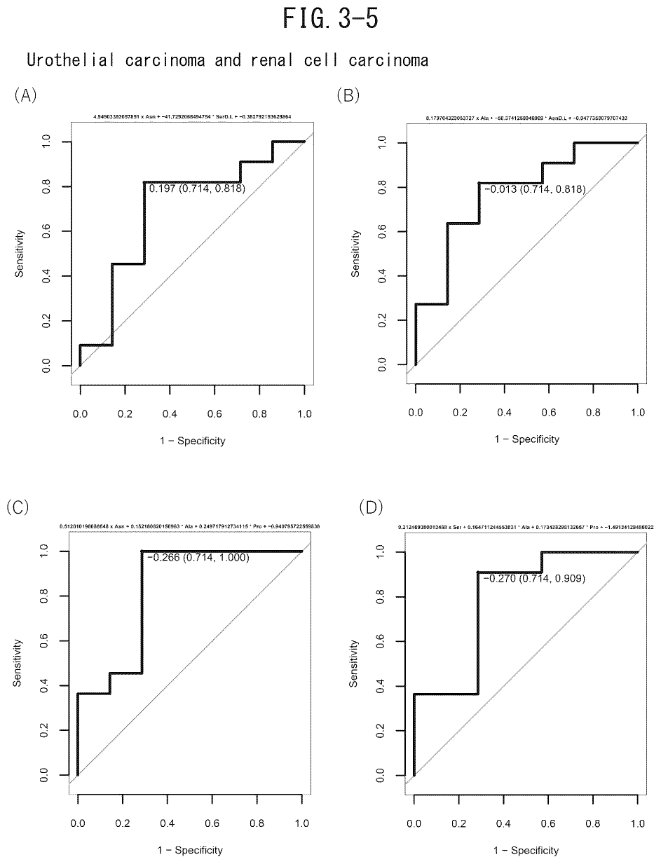

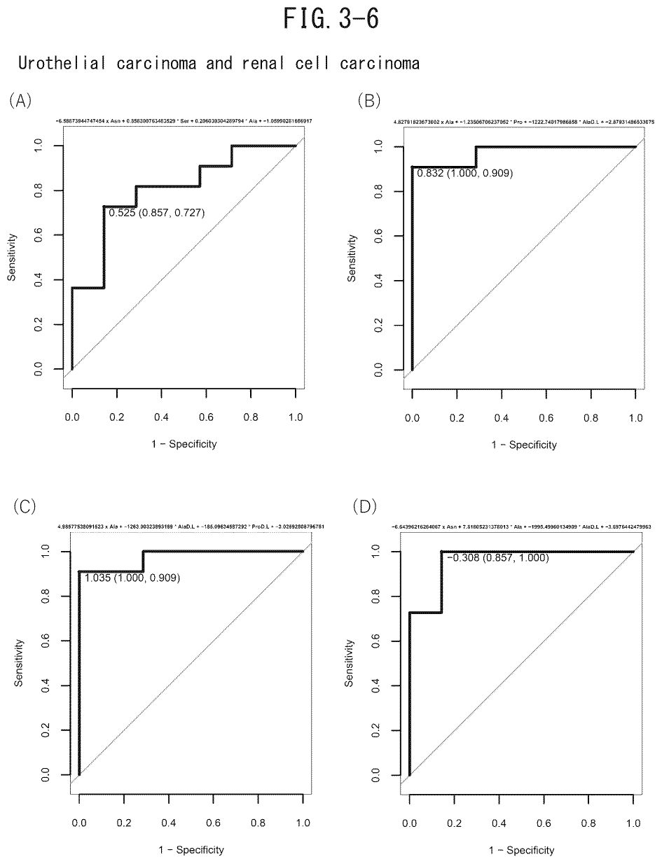

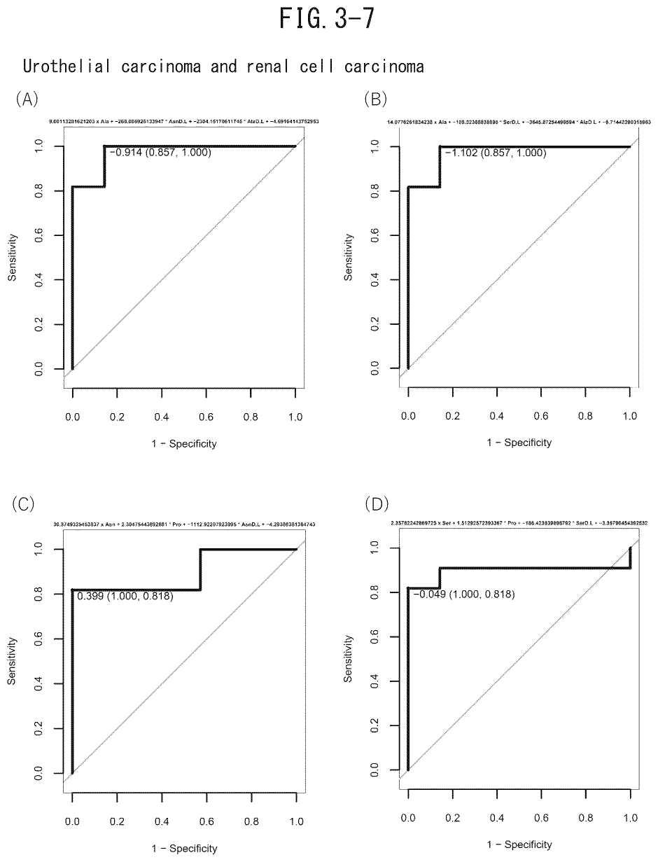

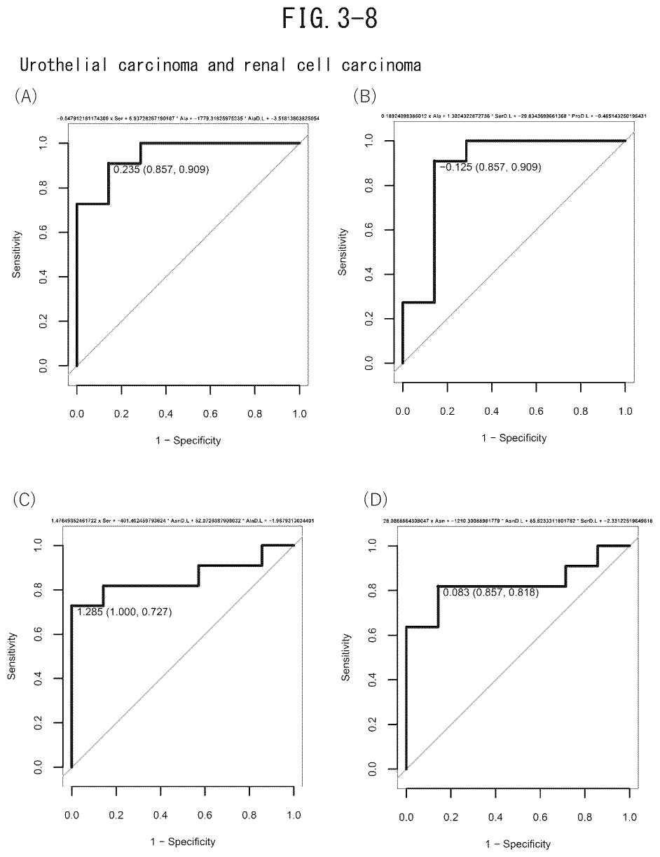

For discrimination between urothelial carcinoma and prostate cancer (FIG. 2-1 to FIG. 2-8), sensitivity was 100% and specificity was 54.5% when the assessment value for PD-Ser was set to 3.695 (FIG. 2-1(A)). When the assessment value for PD-Asn was set to 0.404, sensitivity was 90% and specificity was 63.6% (FIG. 2-1(B)). When the assessment value for PSerD/L was set to 0.030, sensitivity was 90% and specificity was 72.7% (FIG. 2-1(C)). When the assessment value for PAsnD/L was set to 0.007, sensitivity was 80% and specificity was 81.8% (FIG. 2-1(D)). When the assessment value for the prediction formula [−1.05×(PD-Ser)−0.108×(PD-Pro)+3.35] was set to −0.541, sensitivity was 100% and specificity was 63.6% (FIG. 2-2(A)). When the assessment value for the prediction formula [−10.5×(PD-Asn)−0.175×(PD-Pro)+4.13] was set to −0.379, sensitivity was 90% and specificity was 72.7% (FIG. 2-2(B)). When the assessment value for the prediction formula [−1.03×(PD-Ser)−0.136×(PD-Ala)+4.13] was set to −0.232, sensitivity was 90% and specificity was 72.7% (FIG. 2-2(C)). When the assessment value for the prediction formula [−9.99×(PD-Asn)−0.090×(PD-Ala)+4.28] was set to −0.150, sensitivity was 80% and specificity was 81.8% (FIG. 2-2(D)). When the assessment value for the prediction formula [−6.76×(PD-Asn)−0.448×(PD-Ser)+3.86] was set to −0.694, sensitivity was 100% and specificity was 54.5% (FIG. 2-3(A)). When the assessment value for the prediction formula [−115×(PSerD/L)−51.0×(PAlaD/L)+3.94] was set to 0.373, sensitivity was 80% and specificity was 90.9% (FIG. 2-3(B)). When the assessment value for the prediction formula [−8.07×(PD-Asn)−40.7×(PSerD/L)+4.02] was set to −0.546, sensitivity was 100% and specificity was 63.6% (FIG. 2-3(C)). When the assessment value for the prediction formula [−1.14×(PD-Ser)−61.1×(PAlaD/L)+4.61] was set to −0.821, sensitivity was 100% and specificity was 63.6% (FIG. 2-3(D)). When the assessment value for the prediction formula [−1.08×(PD-Ser)−40.0×(PProD/L)+3.59] was set to −0.567, sensitivity was 100% and specificity was 63.6% (FIG. 2-4(A)). When the assessment value for the prediction formula [−11.3×(PD-Asn)−50.9×(PAlaD/L)+5.07] was set to −0.421, sensitivity was 90% and specificity was 72.7% (FIG. 2-4(B)). When the assessment value for the prediction formula [−11.5×(PD-Asn)−68.6×(PProD/L)+4.75] was set to −0.291, sensitivity was 90% and specificity was 72.7% (FIG. 2-4(C)). When the assessment value for the prediction formula [0.006×(PD-Pro)−104×(PSerD/L)+2.66] was set to −0.469, sensitivity was 90% and specificity was 72.7% (FIG. 2-4(D)). When the assessment value for the prediction formula [−105×(PSerD/L)−13.3×(PProD/L)+2.79] was set to −0.429, sensitivity was 90% and specificity was 72.7% (FIG. 2-5(A)). When the assessment value for the prediction formula [−0.126×(PD-Ala)−107×(PSerD/L)+3.62] was set to 0.182, sensitivity was 80% and specificity was 81.8% (FIG. 2-5(B)). When the assessment value for the prediction formula [−0.004×(PD-Pro)−557×(PAsnD/L)+3.83] was set to −0.014, sensitivity was 80% and specificity was 81.8% (FIG. 2-5(C)). When the assessment value for the prediction formula [−521×(PAsnD/L)−26.7×(PAlaD/L)+4.04] was set to −0.104, sensitivity was 80% and specificity was 81.8% (FIG. 2-5(D)). When the assessment value for the prediction formula [−558×(PAsnD/L)−2.67×(PProD/L)+3.84] was set to −0.012, sensitivity was 80% and specificity was 81.8% (FIG. 2-6(A)). When the assessment value for the prediction formula [−5.46×(PD-Asn)−341×(PAsnD/L)+4.36] was set to −0.827, sensitivity was 100% and specificity was 54.5% (FIG. 2-6(B)). When the assessment value for the prediction formula [−0.602×(PD-Ser)−390×(PAsnD/L)+4.55] was set to −0.791, sensitivity was 100% and specificity was 54.5% (FIG. 2-6(C)). When the assessment value for the prediction formula [−1.00×(PD-Ser)−4.16×(PSerD/L)+3.17] was set to −0.677, sensitivity was 100% and specificity was 54.5% (FIG. 2-6(D)). When the assessment value for the prediction formula [−0.051×(PD-Ala)−535×(PAsnD/L)+4.02] was set to −0.236, sensitivity was 80% and specificity was 72 7% (FIG. 2-7(A)). When the assessment value for the prediction formula [−433×(PAsnD/L)−46.0 ×(PSerD/L)+4.18] was set to 0.057, sensitivity was 80% and specificity was 72.7% (FIG. 2-7(B)). When the assessment value for the prediction formula [−7.17×(PD-Asn)−0.437×(PD-Ser)−0.172×(PD-Pro)+4.23] was set to −0.598, sensitivity was 100% and specificity was 63.6% (FIG. 2-7(C)). When the assessment value for the prediction formula [−3.70×(PD-Asn)−0.747×(PD-Ser)−0.123×(PD-Ala)+4.51] was set to −0.297, sensitivity was 90% and specificity was 72.7% (FIG. 2-7(D)). When the assessment value for the prediction formula [−1.04×(PD-Ser)−0.136×(PD-Ala)−0.009×(PD-Pro)+4.15] was set to −0.228, sensitivity was 90% and specificity was 72.7% (FIG. 2-8(A)). When the assessment value for the prediction formula [−10.2×(PD-Asn)−0.086×(PD-Ala)−0.090×(PD-Pro)+4.47] was set to −0.148, sensitivity was 80% and specificity was 81.8% (FIG. 2-8(B)). When the assessment value for the prediction formula [−8.52×(PD-Asn)−0.147×(PD-Pro)−37.9×(PSerD/L)+4.33] was set to −0.482, sensitivity was 100% and specificity was 72.7% (FIG. 2-8(C)). When the assessment value for the prediction formula [−8.17×(PD-Asn)−58.5×(PSerD/L)−53.8×(PAlaD/L)+5.51] was set to −0.585, sensitivity was 100% and specificity was 72.7% (FIG. 2-8(D)).

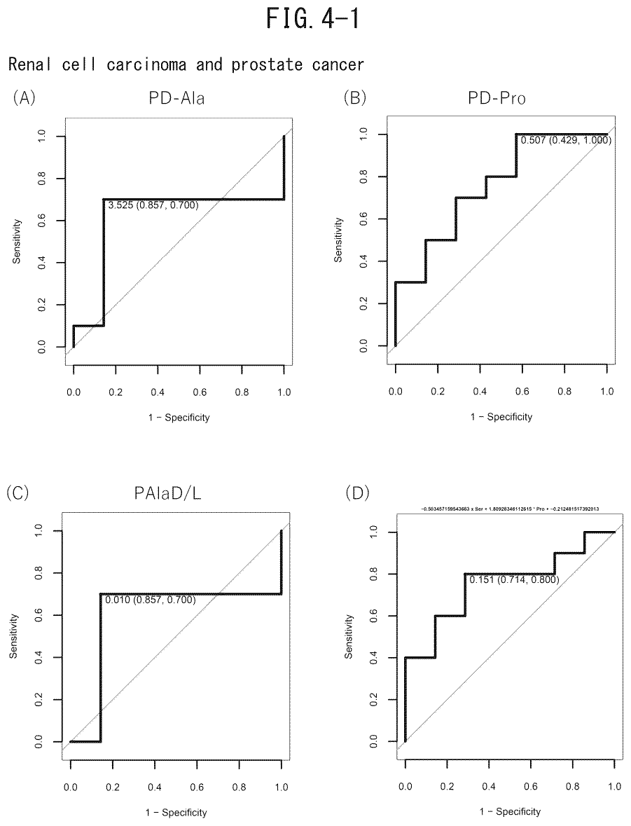

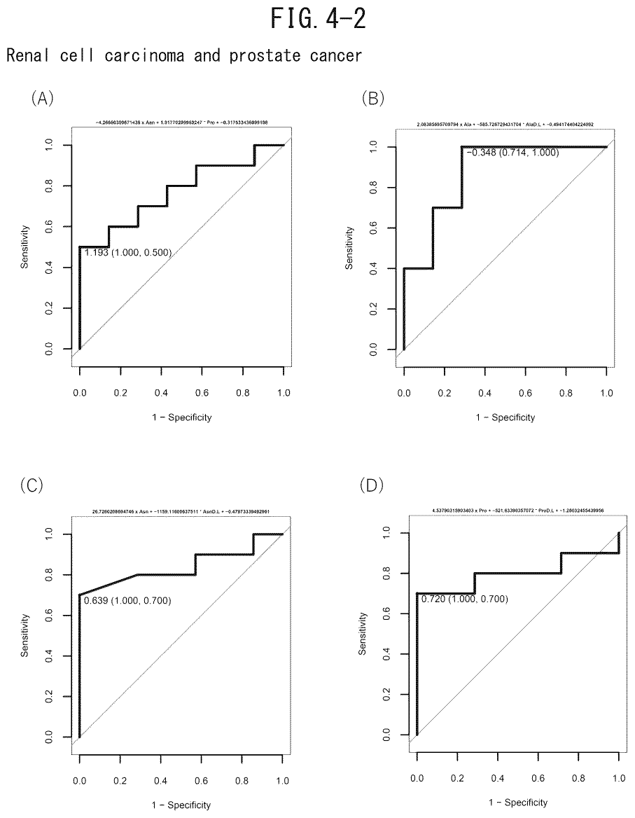

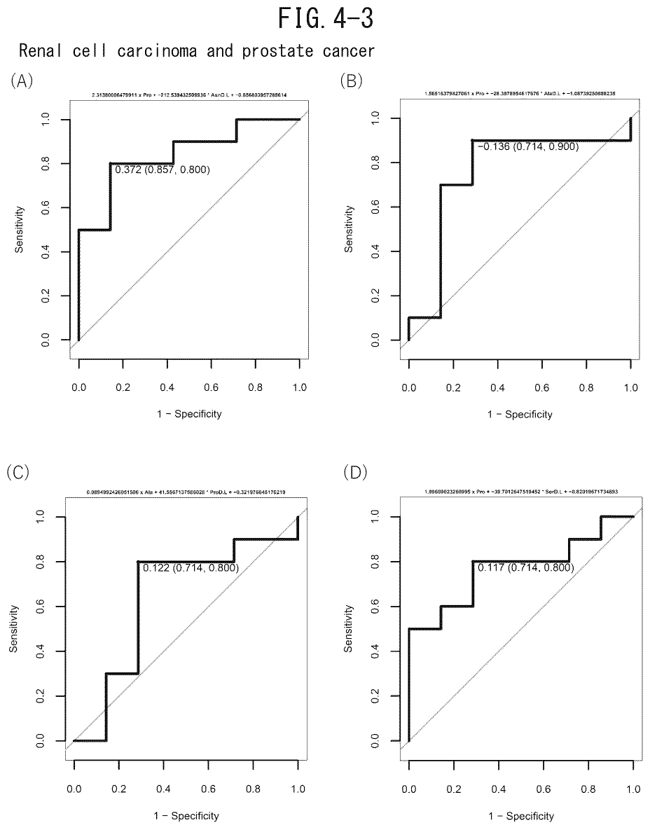

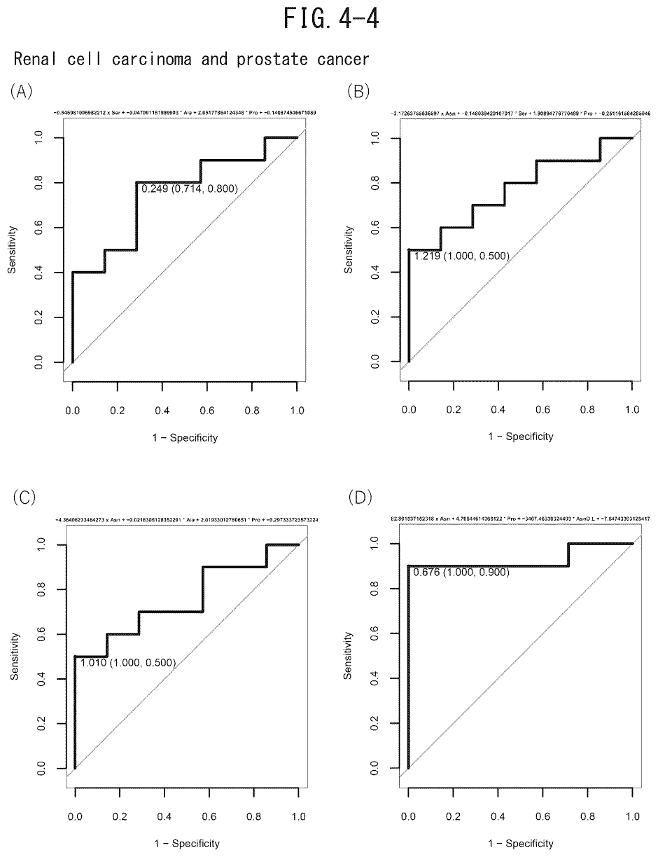

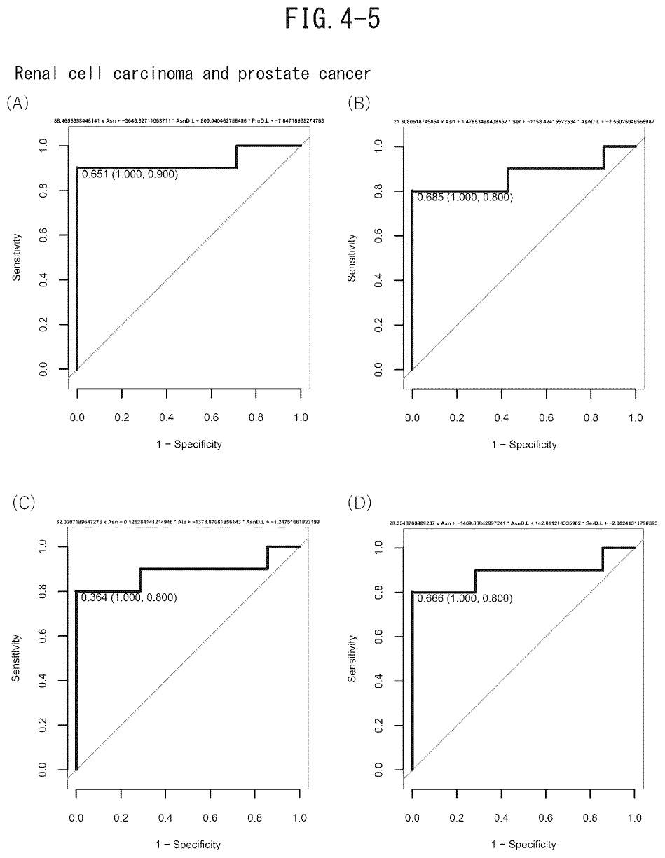

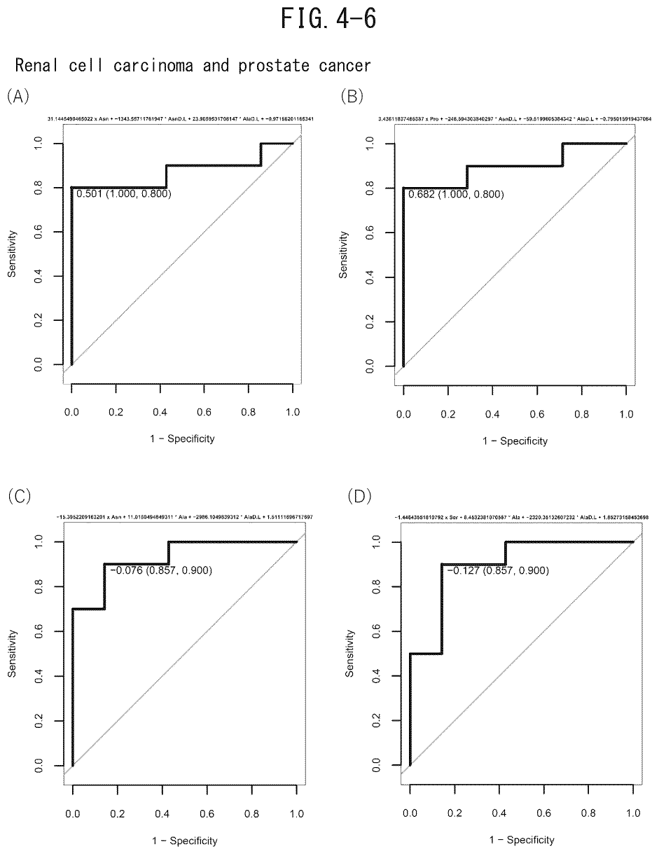

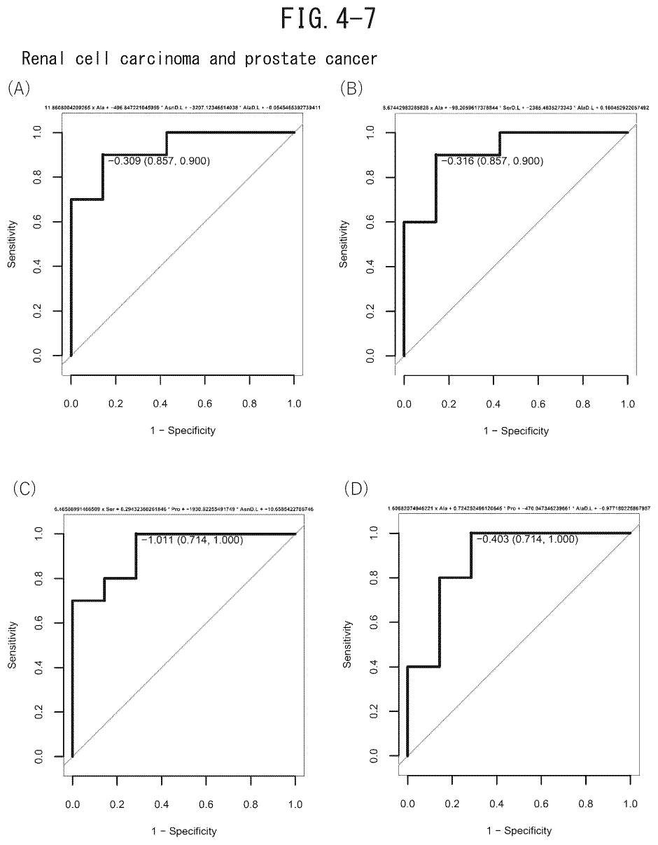

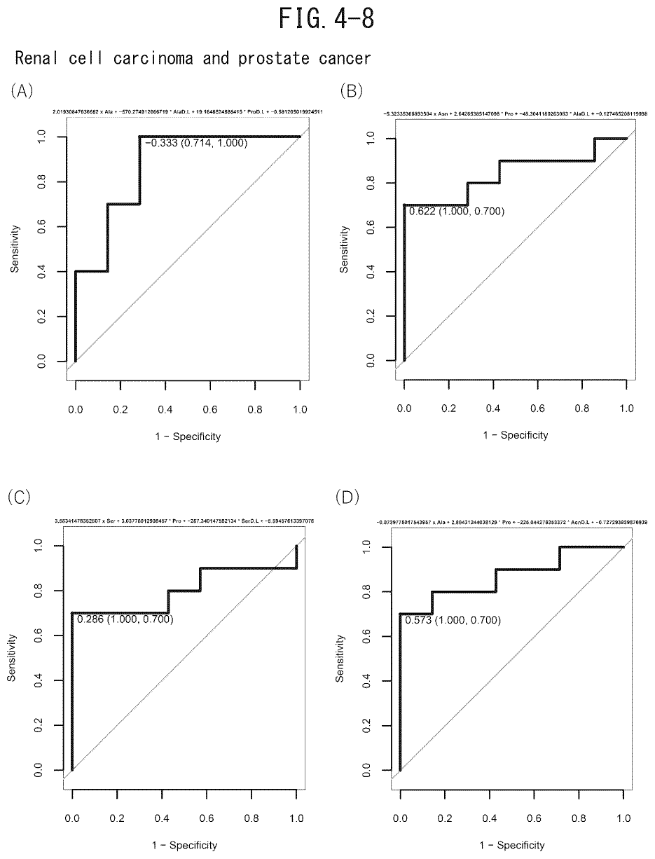

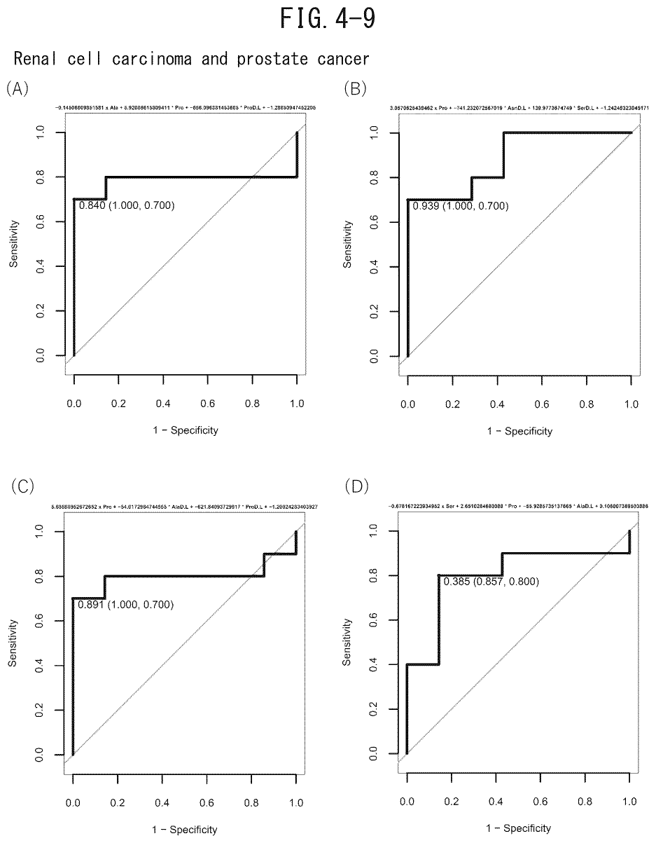

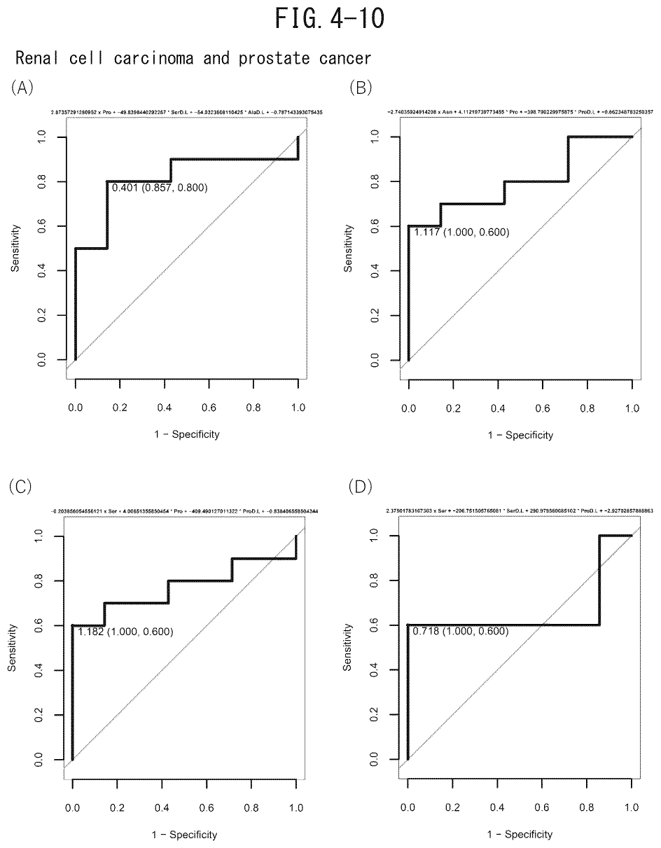

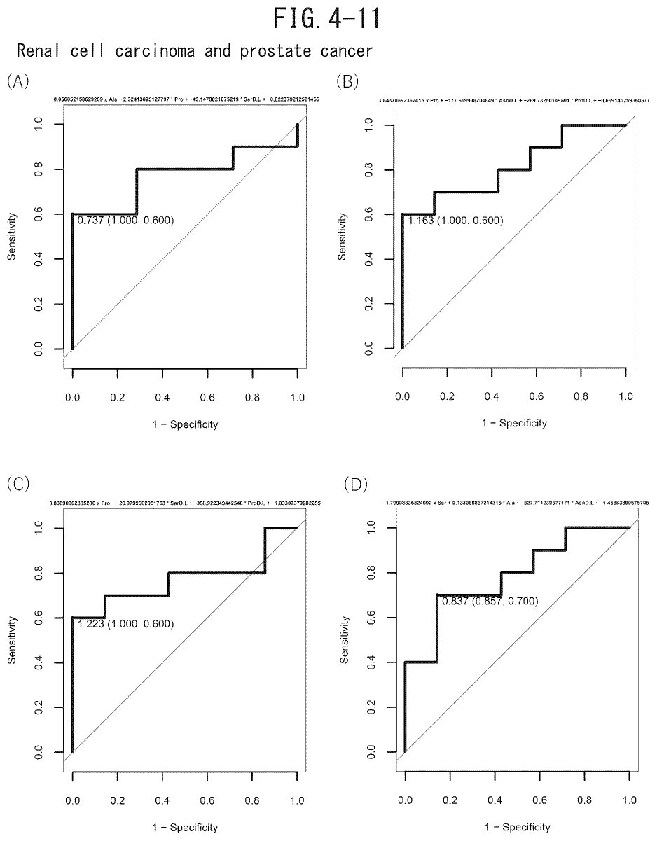

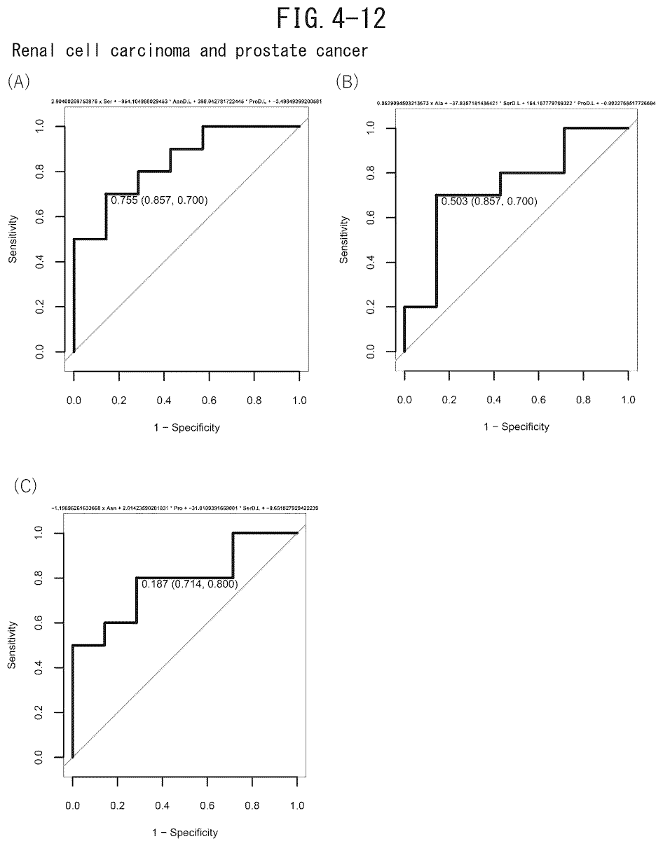

For discrimination between urothelial carcinoma and renal cell carcinoma (FIG. 3), sensitivity was 90.9% and specificity was 71.4% when the assessment value for PD-Asn was set to 0.295 (FIG. 3-1(A)). When the assessment value for PD-Ala was set to 1.850, sensitivity was 100% and specificity was 57.1% (FIG. 3-1(B)). When the assessment value for PD-Ser was set to 2.440, sensitivity was 81.8% and specificity was 71.4% (FIG. 3-1(C)). When the assessment value for PSerD/L was set to 0.030, sensitivity was 72.7% and specificity was 85.7% (FIG. 3-1(D)). When the assessment value for the prediction formula [0.653×(PD-Asn)+0.172×(PD-Ala)−0.834] was set to −0.162, sensitivity was 90.9% and specificity was 71.4% (FIG. 3-2(A)). When the assessment value for the prediction formula [0.225×(PD-Ser)+0.180×(PD-Ala)−1.43] was set to −0.255, sensitivity was 90.9% and specificity was 71.4% (FIG. 3-2(B)). When the assessment value for the prediction formula [0.151×(PD-Ala)+0.274×(PD-Pro)−0.736] was set to −0.162, sensitivity was 90.9% and specificity was 71.4% (FIG. 3-2(C)). When the assessment value for the prediction formula [0.162×(PD-Ser)+0.497×(PD-Pro)−0.683] was set to 0.028, sensitivity was 100% and specificity was 57.1% (FIG. 3-2(D)). When the assessment value for the prediction formula [0.187×(PD-Ala)−26.9×(PProD/L)−0.45] was set to −0.120, sensitivity was 90.9% and specificity was 85.7% (FIG. 3-3(A)). When the assessment value for the prediction formula [4.23×(PD-Ala)−1070×(PAlaD/L)−3.86] was set to −1.143, sensitivity was 100% and specificity was 71.4% (FIG. 3-3(B)). When the assessment value for the prediction formula [0.173×(PD-Ala)−1.68×(PSerD/L)−0.494] was set to −0.099, sensitivity was 81.8% and specificity was 85.7% (FIG. 3-3(C)). When the assessment value for the prediction formula [6.13×(PD-Pro)−810×(PProD/L)−0.792] was set to 0.911, sensitivity was 63.6% and specificity was 100% (FIG. 3-3(D)). When the assessment value for the prediction formula [0.241×(PD-Ser)+36.5×(PAlaD/L)−1.13] was set to −0.071, sensitivity was 90.9% and specificity was 71.4% (FIG. 3-4(A)). When the assessment value for the prediction formula [21.3×(PD-Asn)−733×(PAsnD/L)−1.69] was set to 0.170, sensitivity was 72.7% and specificity was 85.7% (FIG. 3-4(B)). When the assessment value for the prediction formula [0.186×(PD-Ser)+30.1×(PProD/L)−0.419] was set to 0.353, sensitivity was 72.7% and specificity was 85.7% (FIG. 3-4(C)). When the assessment value for the prediction formula [1.62×(PD-Ser)−119×(PSerD/L)−1.50] was set to 0.979, sensitivity was 54.5% and specificity was 100% (FIG. 3-4(D)). When the assessment value for the prediction formula [4.95×(PD-Asn)−41.7×(PSerD/L)−0.383] was set to 0.197, sensitivity was 81.8% and specificity was 71.4% (FIG. 3-5(A)). When the assessment value for the prediction formula [0.180×(PD-Ala)−58.4×(PAsnD/L)−0.048] was set to −0.013, sensitivity was 81.8% and specificity was 71.4% (FIG. 3-5(B)). When the assessment value for the prediction formula [0.512×(PD-Asn)+0.152×(PD-Ala)+0.250×(PD-Pro)−0.941] was set to −0.266, sensitivity was 100% and specificity was 71.4% (FIG. 3-5(C)). When the assessment value for the prediction formula [0.212×(PD-Ser)+0.165×(PD-Ala)+0.173×(PD-Pro)−1.49] was set to −0.270, sensitivity was 90.9% and specificity was 71.4% (FIG. 3-5(D)). When the assessment value for the prediction formula [−6.59×(PD-Asn)+0.858×(PD-Ser)+0.206×(PD-Ala)−1.06] was set to 0.525, sensitivity was 72.7% and specificity was 85.7% (FIG. 3-6(A)). When the assessment value for the prediction formula [4.83×(PD-Ala)−1.24×(PD-Pro)−1220×(PAlaD/L)−2.88] was set to 0.832, sensitivity was 90.9% and specificity was 100% (FIG. 3-6(B)). When the assessment value for the prediction formula [4.99×(PD-Ala)−1260×(PAlaD/L)−185×(PProD/L)−3.03] was set to 1.035, sensitivity was 90.9% and specificity was 100% (FIG. 3-6(C)). When the assessment value for the prediction formula [−6.64×(PD-Asn)+7.82×(PD-Ala)−2000×(PAlaD/L)−3.70] was set to −0.308, sensitivity was 100% and specificity was 85.7% (FIG. 3-6(D)). When the assessment value for the prediction formula [9.00×(PD-Ala)−268×(PAsnD/L)−2300×(PAlaD/L)−4.69] was set to −0.914, sensitivity was 100% and specificity was 85.7% (FIG. 3-7(A)). When the assessment value for the prediction formula [14.1×(PD-Ala)−108×(PSerD/L)−3650×(PAlaD/L)−6.71] was set to −1.102, sensitivity was 100% and specificity was 85.7% (FIG. 3-7(B)). When the assessment value for the prediction formula [30.9×(PD-Asn)−2.30×(PD-Pro)−1110×(PAsnD/L)−4.29] was set to 0.399, sensitivity was 81.8% and specificity was 100% (FIG. 3-7(C)). When the assessment value for the prediction formula [2.36×(PD-Ser)+1.51×(PD-Pro)−188×(PSerD/L)−3.37] was set to −0.049, sensitivity was 81.8% and specificity was 100% (FIG. 3-7(D)). When the assessment value for the prediction formula [−0.548×(PD-Ser)+6.94×(PD-Ala)−1780×(PAlaD/L)−3.52] was set to 0.235, sensitivity was 90.9% and specificity was 85.7% (FIG. 3-8(A). When the assessment value for the prediction formula [0.189×(PD-Ala)+1.30×(PSerD/L)−29.8×(PProD/L)−0.485] was set to −0.125, sensitivity was 90.9% and specificity was 85.7% (FIG. 3-8(B)). When the assessment value for the prediction formula [1.48×(PD-Ser)−401×(PAsnD/L)+52.1×(PAlaD/L)−1.97] was set to 1.285, sensitivity was 72.7% and specificity was 100% (FIG. 3-8(C)). When the assessment value for the prediction formula [28.1×(PD-Asn)−1210×(PAsnD/L)+85.8×(PSerD/L)−2.33] was set to 0.083, sensitivity was 81.8% and specificity was 85.7% (FIG. 3-8(D)).

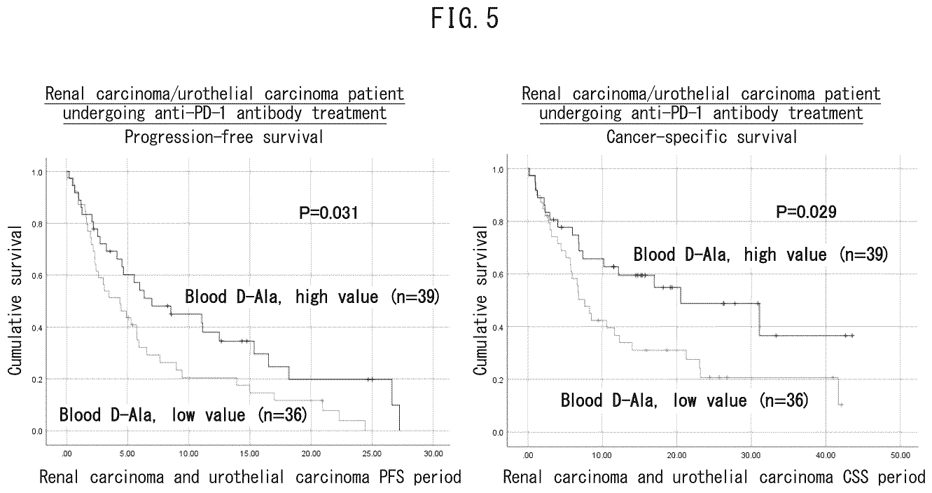

For discrimination between renal cell carcinoma and prostate cancer (FIG. 4), sensitivity was 70.0% and specificity was 85.7% when the assessment value for PD-Ala was set to 3.525 (FIG. 4-1(A)). When the assessment value for PD-Pro was set to 0.507, sensitivity was 100% and specificity was 42.9% (FIG. 4-1(B)). When the assessment value for PAlaD/L was set to 0.010, sensitivity was 70.0% and specificity was 85.7% (FIG. 4-1(C)). When the assessment value for the prediction formula [−0.503×(PD-Ser)+1.81×(PD-Pro)−0.212] was set to 0.151, sensitivity was 80.0% and specificity was 71.4% (FIG. 4-1(D)). When the assessment value for the prediction formula [−4.27×(PD-Asn)+1.92×(PD-Pro)−0.318] was set to 1.193, sensitivity was 50.0% and specificity was 100% (FIG. 4-2(A)). When the assessment value for the prediction formula [2.08×(PD-Ala)−586×(PAlaD/L)−0.494] was set to −0.348, sensitivity was 100% and specificity was 71.4% (FIG. 4-2(B)). When the assessment value for the prediction formula [26.7×(PD-Asn)−1160×(PAsnD/L)−0.480] was set to 0.639, sensitivity was 70% and specificity was 100% (FIG. 4-2(C)). When the assessment value for the prediction formula [4.54×(PD-Pro)−522×(PProD/L)−1.28] was set to 0.720, sensitivity was 70% and specificity was 100% (FIG. 4-2(D)). When the assessment value for the prediction formula [2.31×(PD-Pro)−213×(PAsnD/L)−0.657] was set to 0.372, sensitivity was 80% and specificity was 85.7% (FIG. 4-3(A)). When the assessment value for the prediction formula [1.57×(PD-Pro)−28.4×(PAlaD/L)−1.09] was set to −0.136, sensitivity was 90% and specificity was 71.4% (FIG. 4-3(B)). When the assessment value for the prediction formula [0.089×(PD-Ala)+41.6×(PProD/L)−0.322] was set to 0.122, sensitivity was 80% and specificity was 71.4% (FIG. 4-3(C)). When the assessment value for the prediction formula [2.00×(PD-Pro)−39.7×(PSerD/L)+0.820] was set to −0.117, sensitivity was 80% and specificity was 71.4% (FIG. 4-3(D)). When the assessment value for the prediction formula [−0.545×(PD-Ser)−0.047×(PD-Ala)+2.05×(PD-Pro)−0.141] was set to 0.249, sensitivity was 80% and specificity was 71.4% (FIG. 4-4(A)). When the assessment value for the prediction formula [−3.17×(PD-Asn)−0.148×(PD-Ser)+1.91×(PD-Pro)−0.251] was set to 1.219, sensitivity was 50% and specificity was 100% (FIG. 4-4(B)). When the assessment value for the prediction formula [−4.36×(PD-Asn)−0.022×(PD-Ala)+2.02×(PD-Pro)−0.297] was set to 1.010, sensitivity was 50% and specificity was 100% (FIG. 4-4(C)). When the assessment value for the prediction formula [82.8×(PD-Asn)+4.71×(PD-Pro)−3410×(PAsnD/L)−7.65] was set to 0.676, sensitivity was 90% and specificity was 100% (FIG. 4-4(D)). When the assessment value for the prediction formula [88.5×(PD-Asn)−3650×(PAsnD/L)+800×(PProD/L)−7.85] was set to 0.651, sensitivity was 90% and specificity was 100% (FIG. 4-5(A)). When the assessment value for the prediction formula [21.3×(PD-Asn)+1.48×(PD-Ser)−1160×(PAsnD/L)−2.55] was set to 0.685, sensitivity was 80% and specificity was 100% (FIG. 4-5(B)). When the assessment value for the prediction formula [32.0×(PD-Asn)+0.125×(PD-Ala)−1370×(PAsnD/L)−1.25] was set to 0.364, sensitivity was 80% and specificity was 100% (FIG. 4-5(C)). When the assessment value for the prediction formula [28.3×(PD-Asn)−1470×(PAsnD/L)+142×(PSerD/L)−2.00] was set to 0.666, sensitivity was 80% and specificity was 100% (FIG. 4-5(D)). When the assessment value for the prediction formula [31.1×(PD-Asn)−1340×(PAsnD/L)+23.9×(PAlaD/L)−0.972] was set to 0.501, sensitivity was 80% and specificity was 100% (FIG. 4-6(A)). When the assessment value for the prediction formula [3.44×(PD-Pro)−247×(PAsnD/L)−59.5×(PAlaD/L)−0.795] was set to 0.682, sensitivity was 80% and specificity was 100% (FIG. 4-6(B)). When the assessment value for the prediction formula [−15.4×(PD-Asn)+11.0×(PD-Ala)−2990×(PAlaD/L)+1.51] was set to −0.076, sensitivity was 90% and specificity was 85.7% (FIG. 4-6(C)). When the assessment value for the prediction formula [−1.45×(PD-Ser)+8.45×(PD-Ala)−2320×(PAlaD/L)−1.85] was set to −0.127, sensitivity was 90% and specificity was 85.7% (FIG. 4-6(D)). When the assessment value for the prediction formula [11.9×(PD-Ala)−497×(PAsnD/L)−3210×(PAlaD/L)−0.055] was set to −0.309, sensitivity was 90% and specificity was 85.7% (FIG. 4-7(A)). When the assessment value for the prediction formula [8.67×(PD-Ala)−98.2×(PSerD/L)−2370×(PAlaD/L)+0.160] was set to −0.316, sensitivity was 90% and specificity was 85.7% (FIG. 4-7(B)). When the assessment value for the prediction formula [6.47×(PD-Ser)+6.29×(PD-Pro)−1930×(PAsnD/L)−10.7] was set to −1.011, sensitivity was 100% and specificity was 71.4% (FIG. 4-7(C)). When the assessment value for the prediction formula [1.61×(PD-Ala)+0.724×(PD-Pro)−470×(PAlaD/L)−0.977] was set to −0.403, sensitivity was 100% and specificity was 71.4% (FIG. 4-7(D)). When the assessment value for the prediction formula [2.02×(PD-Ala)−570×(PAlaD/L)+19.2×(PProD/L)−0.581] was set to −0.333, sensitivity was 100% and specificity was 71.4% (FIG. 4-8(A)). When the assessment value for the prediction formula [−5.32×(PD-Asn)+2.64×(PD-Pro)−48.3×(PAlaD)/L)−0.127] was set to 0.622, sensitivity was 70% and specificity was 100% (FIG. 4-8(B)). When the assessment value for the prediction formula [3.58×(PD-Ser)+3.04×(PD-Pro)−287×(PSerD/L)−5.59] was set to 0.286, sensitivity was 70% and specificity was 100% (FIG. 4-8(C)). When the assessment value for the prediction formula [−0.074×(PD-Ala)+2.80×(PD-Pro)−225×(PAsnD/L)−0.727] was set to 0.573, sensitivity was 70% and specificity was 100% (FIG. 4-8(D)). When the assessment value for the prediction formula [−0.145×(PD-Ala)+5.92×(PD-Pro)−656×(PProD/L)−1.29] was set to 0.840, sensitivity was 70% and specificity was 100% (FIG. 4-9(A)). When the assessment value for the prediction formula [3.06×(PD-Pro)−741×(PAsnD/L)+140×(PSerD/L)−1.24] was set to 0.939, sensitivity was 70% and specificity was 100% (FIG. 4-9(B)). When the assessment value for the prediction formula [5.69×(PD-Pro)−54.0×(PAlaD/L)−622×(PProD/L)−1.20] was set to 0.891, sensitivity was 70% and specificity was 100% (FIG. 4-9(C)). When the assessment value for the prediction formula [−0.678×(PD-Ser)+2.65×(PD-Pro)−55.9×(PAlaD/L)+0.106] was set to 0.385, sensitivity was 80% and specificity was 85.7% (FIG. 4-9(D)). When the assessment value for the prediction formula [2.87×(PD-Pro)−49.8×(PSerD/L)−54.0×(PAlaD/L)−0.787] was set to 0.401, sensitivity was 80% and specificity was 85.7% (FIG. 4-10(A)). When the assessment value for the prediction formula [−2.74×(PD-Asn)+4.11×(PD-Pro)−399×(PProD/L)−0.662] was set to 1.117, sensitivity was 60% and specificity was 100% (FIG. 4-10(B)). When the assessment value for the prediction formula [−0.204×(PD-Ser)+4.01×(PD-Pro)−409×(PProD/L)−0.838] was set to 1.182, sensitivity was 60% and specificity was 100% (FIG. 4-10(C)). When the assessment value for the prediction formula [2.38×(PD-Ser)−207×(PSerD/L)+291×(PProD/L)−2.93] was set to 0.718, sensitivity was 60% and specificity was 100% (FIG. 4-10(D)). When the assessment value for the prediction formula [−0.056×(PD-Ala)+2.32×(PD-Pro)−43.1×(PSerD/L)−0.822] was set to 0.737, sensitivity was 60% and specificity was 100% (FIG. 4-11(A)). When the assessment value for the prediction formula [3.64×(PD-Pro)−172×(PAsnD/L)−270×(PProD/L)−0.689] was set to 1.163, sensitivity was 60% and specificity was 100% (FIG. 4-11(B)). When the assessment value for the prediction formula [3.84×(PD-Pro)−20.1×(PSerD/L)−359×(PProD/L)−1.03] was set to 1.223, sensitivity was 60% and specificity was 100% (FIG. 4-11(C)). When the assessment value for the prediction formula [1.80×(PD-Ser)+0.134×(PD-Ala)−528×(PAsnD/L)−1.46] was set to 0.837, sensitivity was 70% and specificity was 85.7% (FIG. 4-11(D)). When the assessment value for the prediction formula [2.90×(PD-Ser)−954×(PAsnD/L)+398×(PProD/L)−3.50] was set to 0.755, sensitivity was 70% and specificity was 85.7% (FIG. 4-12(A)). When the assessment value for the prediction formula [0.063×(PD-Ala)−37.8×(PSerD/L)+164×(PProD/L)−0.002] was set to 0.503, sensitivity was 70% and specificity was 85.7% (FIG. 4-12(B)). When the assessment value for the prediction formula [−1.20×(PD-Asn)+2.01×(PD-Pro)−31.0×(PSerD/L)−0.652] was set to −0.187, sensitivity was 80% and specificity was 71.4% (FIG. 4-12(C)).

These results demonstrated that using markers relating to the amount of the D-amino acids in the blood of subjects can simultaneously provide information for detection of cancer in the subjects and discrimination of cancer type, in a single examination.

### (3) Assessment of Treatment Responsiveness by PD-AA

FIG. 5 shows the results of analyzing progression-free survival (PFS) and cancer-specific survival (CSS) by Kaplan-Meier analysis for 2 groups: a high PD-Ala value group (n=39) and a low PD-Ala value group (n=36), among patients with renal carcinoma and urothelial carcinoma that had received anti-cancer treatment with an anti-PD-1 antibody formulation in the control cohort.

The high PD-Ala value group all had higher PFS and CSS survival rates compared to the low-value group, and were evaluated as having successful, good or high anticancer agent treatment responsiveness. The low PD-Ala value group was evaluated as having unsuccessful, poor or low anticancer agent treatment responsiveness (PFS: P=0.031, CSS: P=0.029). The knowledge that prognosis is satisfactory in a high PD-Ala value group among cancer patients suggests that increasing D-Ala is one biological defense function against cancer.

Since it is possible to assess the presence or absence of cancer and discrimination of cancer type according to (1) and (2) above, and also to assess treatment responsiveness according to (3), it is possible to use one or more markers relating to the amounts of the D-amino acids in blood to provide multi-level information on cancer in a subject in either a simultaneous or successive manner. As one specific example, it is possible to assess the presence or absence cancer by PD-Asn, PD-Ser, PD-Ala or PD-Pro, as a first stage, to assess the cancer type (such as urothelial carcinoma) by PD-Asn or PD-Ser, as the second stage, and to assess treatment responsiveness to anticancer agents by PD-Ala, as the third stage, thus aiding in selection of specific anti-cancer treatments.

Thus, by using markers relating to the amounts of the D-amino acids in the blood of the subject it is possible assess the reaction and therapeutic effect of a subject to an anticancer agent, and to provide information regarding treatment responsiveness.

1. A method for providing information regarding cancer type of a subject, comprising:
assessing the cancer type of the subject using a marker relating to the amount of a D-amino acid in blood of the subject; and
providing information relating to the cancer type of the subject, based on the assessment results.

2. The method according to claim 1, wherein:
the marker relating to the amount of a D-amino acid in blood of the subject is used to assess the cancer type and treatment responsiveness of the subject, and
the information relating to the cancer type and treatment responsiveness of the subject is provided, based on the assessment results.

3. The method according to claim 1, wherein the marker relating to the amount of a D-amino acid is a formula or value obtained by calibrating the amount of the D-amino acid with an in vivo substance of the subject.

4. The method according to claim 3, wherein the in vivo substance is an L-amino acid.

5. The method according to claim 1, wherein the marker relating to the amount of a D-amino acid is a formula or value obtained by calibrating the amount of the D-amino acid with a marker relating to kidney function in the subject.

6. The method according to claim 5, wherein the marker relating to kidney function is the amount of one or more factors selected from the group consisting of creatinine, cystatin C, inulin clearance, creatinine clearance, D-amino acid clearance, urine protein, urine albumin, β2-MG, α1-MG, NAG, L-FABP and NGAL.

7. The method according to claim 1, wherein the D-amino acid is one or more selected from the group consisting of D-proline, D-serine, D-alanine and D-asparagine.

8. The method according to claim 1, wherein the cancer is urogenital cancer.

9. The method according to claim 8, wherein the urogenital cancer is cancer selected from the group consisting of renal cell carcinoma, renal pelvis/ureter cancer, bladder cancer and prostate cancer.

10. The method according to claim 8, wherein the urogenital cancer is cancer selected from the group consisting of clear cell carcinoma, urothelial carcinoma, adenocarcinoma and seminoma.

11. The method according to claim 1, wherein the assessment is made by comparing the marker relating to the amount of a D-amino acid with an assessment value for cancer type determined from the amount of the D-amino acid in the blood of subjects with cancer.

12. A system for carrying out the method according to claim 1, the system including a memory unit, an input unit, an analytical measurement unit, a data processing unit and an output unit, and the system being configured so that:
the input unit inputs information from a subject,
the analytical measurement unit acquires a marker relating to the amount of a D-amino acid of the subject by analytical measurement of information from the subject, inputted through the input unit,
the memory unit stores an assessment value related to cancer type,
the data processing unit assesses the cancer type of the subject by comparing the marker of the subject acquired by the analytical measurement unit, with the assessment value stored in the memory unit, and
the output unit outputs the assessment results from the data processing unit as information relating to the cancer type of the subject.

13. The system according to claim 12, which is further configured so that:
the memory unit stores an assessment value relating to cancer type and treatment responsiveness,
the data processing unit assesses the cancer type and treatment responsiveness of the subject by comparing the marker of the subject acquired by the analytical measurement unit with the assessment value stored in the memory unit, and
the output unit outputs the assessment results from the data processing unit as information relating to the cancer type and treatment responsiveness of the subject.
